(19) United States
(12) Patent Application Publication
(10) Pub. No.: US 2020/0378954 A1
(43) Pub. Date: Dec. 3, 2020

(54) DETECTING CYTOKINE SIGNALING RESPONSIVENESS IN IMMUNE CELLS

(71) Applicant: City of Hope, Duarte, CA (US)

(72) Inventors: Peter P. LEE, San Marino, CA (US); Lei WANG, Fullerton, CA (US)

(21) Appl. No.: 16/889,402

(22) Filed: Jun. 1, 2020

Related U.S. Application Data
(60) Provisional application No. 62/855,681, filed on May 31, 2019.

(51) Int. Cl. G01N 33/50; G01N 33/68; A61K 31/675; A61K 31/519; A61K 31/513; A61K 31/704; A61K 31/015

(52) U.S. Cl. CPC G01N 33/5091; G01N 33/6863; A61K 31/675; A61K 31/015; A61K 31/513; A61K 31/704; A61K 31/519

(57) ABSTRACT

Provided herein are methods of detecting cytokine signaling responsiveness in immune cells from a cancer subject and determining risk of relapse of cancer in a subject. The methods include isolating cells from a blood sample from the cancer subject thereby forming an isolated blood cell fraction that includes isolated blood sample cells, mixing the isolated blood sample cells with a cytokine, where the cytokine is selected from TGFβ, IL-10, IL-4 and IFNγ, and detecting the responsiveness of the isolated blood sample cells to the cytokine.

### CROSS REFERENCED APPLICATIONS

This application claims priority to U.S. Ser. No. 62/855,681, filed May 31, 2019 and is incorporated herein in its entirety.

### STATEMENT AS TO RIGHTS TO INVENTIONS MADE UNDER FEDERALLY SPONSORED RESEARCH AND DEVELOPMENT

This invention was made with government support under grant no. P30CA33572 awarded by the National Cancer Institutes of Health and W81XWH-12-1-0366 from Department of Defense Breast Cancer Research Program. The government has certain rights in the invention.

### BACKGROUND

Regulatory T (Treg) cells play a major role in the development of an immunosuppressive tumor microenvironment. The origin of intratumoral Treg cells and their relationship with peripheral blood Treg cells remains unclear. Treg cells comprise of at least three functionally distinct subpopulations.

Forkhead box P3 (FoxP3)⁺ regulatory T (Treg) cells are potent suppressors of effector immune cells and essential to maintain immunological tolerance and homeostasis. A broad array of immunosuppressive mechanisms are used by Treg cells to regulate immune responses. Treg cells compete for T cell growth factor IL-2 via expression of high-affinity IL-2 receptor complexes composed of CD25 (IL-2Rα), CD122 (IL-2Rβ) and CD132 (IL-2Rγ) to inhibit effector T cell proliferation. Treg cells also exert direct suppressive activity via secreting immunosuppressive cytokines such as transforming growth factor (TGF)-β and IL-10. In addition to expressing cytotoxic T-lymphocyte-associated protein (CTLA)-4, Treg cells also express ATP metabolizing enzymes CD39 and CD73 to convert extracellular ATP released by dying cells to adenosine, which has various immunomodulatory effects. Moreover, Treg cells may exert direct cytotoxic effect on effector cells via granzyme secretion. (See for example References 1-6).

In cancer, Treg cells are potent inhibitors of antitumor immunity and contribute to the development of an immunosuppressive tumor microenvironment (TME). High intratumoral Treg cells in particular, Treg/CD8+ T cell ratio, is often associated with unfavorable prognosis in various types of cancer.

### BRIEF SUMMARY

In an aspect, provided herein are methods of detecting cytokine signaling responsiveness in immune cells from a cancer subject. The methods include isolating cells from a blood sample from the cancer subject thereby forming an isolated blood cell fraction that includes isolated blood sample cells, mixing the isolated blood sample cells with a cytokine, where the cytokine is selected from TGFβ, IL-10, IL-4 and IFNγ, and detecting the responsiveness of the isolated blood sample cells to the cytokine.

In an aspect, provided herein are methods of determining risk of relapse of cancer in a subject. The methods include isolating cells from a blood sample from the subject thereby forming an isolated blood cell fraction that includes isolated blood sample cells, mixing the isolated blood sample cells with a cytokine, where the cytokine is selected from TGFβ, IL-10, IL-4 or IFNγ, detecting the responsiveness of the isolated blood sample cells to the cytokine, and determining whether said subject has a high risk of cancer relapse.

In an aspect, provided herein are methods of treating a cancer in a subject in need thereof. The methods include isolating cells from a blood sample from the subject thereby forming an isolated blood cell fraction comprising isolated blood sample cells, mixing the isolated blood sample cells with a cytokine, where the cytokine is selected from TGFβ, IL-10, IL-4 and IFNγ, detecting the responsiveness of the isolated blood sample cells to the cytokine, and treating said subject with a therapeutic regimen.

In an aspect, provided herein is a method of preparing a sample including isolating cells from a blood sample from a subject thereby forming an isolated blood cell fraction including isolated blood sample cells, mixing the isolated blood sample cells with a cytokine, wherein said cytokine is selected from TGFβ, IL-10, IL-4 and IFNγ, detecting the responsiveness of the isolated blood sample cells to said cytokine, and quantifying an amount of responsiveness of the isolated blood sample cells to said cytokine.

### BRIEF DESCRIPTION OF THE DRAWINGS

FIGS. 1A-1G demonstrate that IL-2 not only induces higher STAT5 but also STAT1 and STAT3 phosphorylation in peripheral blood Treg 1 cell subpopulation from patients with breast cancer (BC). FIG. 1A are representative flow plots showing peripheral blood Treg cell subpopulation I (CD4⁺CD45RA⁺FoxP3^{lo}), II (CD4⁺CD45RA⁻FoxP3^{hi}) and III (CD4⁺CD45RA⁻ FoxP3^{lo}) from newly diagnosed patients with BC (n=118) and intrI aatumoral Treg cells from paired untreated primary breast tumors (n=28). FIG. 1B is an average composition of peripheral blood Treg cell subpopulation I, II and III from patients with BC at diagnosis (n=118). FIGS. 1C-1D are representative flow plots and the percentage of CD25⁺ in peripheral Tconv (CD4⁺FoxP3⁻), Treg I, II, III cells (n=40) and paired intratumoral Treg cells (n=14) from patients with BC, ****p<0.0001. FIGS. 1E-1F show the percentage of IL-2Rβ+, ****p<0.0001 (FIG. 1E) or IL-2Rγ+ (n=20), ****p<0.0001; ***p=0.0007; **p=0.003 (FIG. 1F) in peripheral Tconv, Treg I, II and III cells from patients with BC. FIG. 1G is data showing PBMCs from newly diagnosed patients with BC (n=40) were stimulated with IL-2 (100 U/ml, 15 mins) and IL-2-induced phosphorylation of STAT5, STAT1 and STAT3 in peripheral Tconv, Treg I, II and III cells were determined by phosphoflow cytometry with anti-p-STAT5 (pY694), p-STAT1 (pY701) and p-STAT3 (pY705) antibodies. Cytokine signaling responsiveness is represented by ΔMFI, which is cytokine stimulated MFI minus unstimulated MFI of p-STAT1/3/5. ****p<0.0001; *p<0.05, Friedman test. Shown are mean±s.e.m Data from intratumoral Treg cells were highlighted with purple box.

FIGS. 2A-2D demonstrate immune phenotype of peripheral blood Treg II cells is similar to paired intratumoral Treg cells. FIG. 2A demonstrates the gating strategy used to isolate CD45RA⁺ FoxP3^{lo} Treg I cells, CD45RA⁻FoxP3^{hi} Treg II cells, and CD45RA⁻FoxP3^{lo} Treg III cells. FIG. 2B are representative flow plots for percentage of IL-2Rβ+ amongst all Treg subpopulations. FIG. 2C are representative flow plots for levels of IL-2Rγ amongst all Treg subpopulations. FIG. 2D shows the gating strategy for examined IL-2-induced STAT1 and STAT3 phosphorylation (p-STAT1/3) in peripheral Treg cell subpopulations.

FIGS. 3A-3F are representative flow plots with percentages demonstrating that peripheral blood Treg II cells have similar immune phenotypes with intratumoral Treg cells. Representative flow plots and the percentages of CD39+, ****p<0.0001; **p=0.006 (FIG. 3A), CTLA4+(intracellularly stained), ****p<0.0001 (FIG. 3B), TIGIT+, ****p<0.0001; **p=0.003 (FIG. 3C), ICOS+, ****p<0.0001; **p=0.002 (FIG. 3D), OX40+, ****p<0.0001 (FIG. 3E) or Helios+, ****p<0.0001 (FIG. 3F) in peripheral Tconv, Treg I, II and III cells from newly diagnosed patients with BC (n=40) and in paired intratumoral Treg cells from untreated primary breast tumors (n=8) were determined by flow cytometry. Friedman test. Shown are mean±s.e.m. Data from intratumoral Treg cells were highlighted with purple box.

FIGS. 4A-4F present data showing expressions of immune-regulating proteins in peripheral blood Treg cell subpopulations and intratumoral Treg cells. The percentages of CD73+ (n=40) (FIG. 4A), PD1+ (n=40), ****p<0.0001 (FIG. 4B), Tim3+ (n=40), ****p<0.0001 (FIG. 4C), LAG3+ (n=40), ****p<0.0001; ***p=0.0002 (FIG. 4D), GITR+ (n=20), ****p<0.0001; **p=0.007 (FIG. 4E) or HLA-DR+ (n=40), ****p<0.0001; ***p=0.0003 (FIG. 4F) in peripheral Tconv, Treg I, II and III cells from newly diagnosed patients with BC were determined by flow cytometry. Friedman test. Shown are mean±s.e.m. Representative flow plots showing the percentages of LAG3+ (FIG. 4D), GITR+ (FIG. 4E), or HLA-DR+ (FIG. 4F) in intratumor Treg cells from untreated primary breast tumors (n=8). Data from intratumoral Treg cells were highlighted with purple box.

FIGS. 5A-5G show that peripheral blood Treg II cells have similar chemokine receptor expression patterns with intratumoral Treg cells. FIGS. 5A-5B are representative flow plots and the percentages of CCR8⁺ in peripheral Tconv, Treg I, II and III cells from newly diagnosed patients with breast cancer (BC) (n=40), ****p<0.0001; ***p=0.0006 (FIG. 5A) and in paired intratumoral Treg cells from untreated primary breast tumors (n=13) (FIG. 5B). FIG. 5C presents flow cytometry sorted peripheral Tconv, Treg I, II and III cells (n=10) that were left to migrate through 3 μm-pore transwells in response to CCL1 (100 ng/ml). Results are expressed as percentage of migrated cells after 3 hr, ****p<0.0001; **p=0.002; *p=0.022. FIG. 5D shows MFI ratio of protein expression by intratumoral CCR8⁺ vs CCR8− Treg cells. FIGS. 5E-5G show the percentages and representative flow plots of CCR4⁺, ****p<0.0001; *p=0.03 (FIG. 5E), CCR5+, ****p<0.0001; **p=0.009 (FIG. 5F), CXCR6⁺, ****p<0.0001; *p=0.043 (FIG. 5G) in peripheral Tconv, Treg I, II and III cells from new diagnosed patients with BC (n=20) and intratumoral Tregs from untreated primary breast tumors (n=8). Friedman test. Shown are mean±s.e.m. Data from intratumoral Treg cells were highlighted with purple box.

FIG. 6 shows representative flow plots showing the flow sort gating strategy of peripheral blood Tconv and Treg cell subpopulations. Peripheral T_{reg} I (CD45RA⁺CD25^{lo}), II (CD45RA⁻ CD25^{hi}), III (CD45RA⁻CD25^{lo}) and T_{conv} (CD45RA⁻CD25⁻) cells were sorted from PBMCs of newly diagnosed patients with BC.

FIGS. 7A-7C demonstrate the differential expression of chemokine receptors CCR2, CCR10 and CXCR3 in intratumoral Treg cells. The percentages and representative flow plots of CCR2+, ***p=0.0003; *p=0.03 (FIG. 7A), CCR10+, ****p<0.0001; **p=0.009 (FIG. 7B), CXCR3+, ****p<0.0001; *p=0.025 (FIG. 7C) in peripheral Tconv, Treg I, II and III cells from newly diagnosed patients with BC (n=20) and in intratumor Treg cells from untreated primary breast tumors (n=8). Friedman test. Shown are mean±s.e.m. Data from intratumoral Treg cells were highlighted with purple box.

FIGS. 8A-8D is data demonstrating that intratumoral Treg cells share more clonal overlap with peripheral blood Treg II cells. TCRβ CDR3 regions of peripheral Treg I, II and III cells, which were sorted from PBMCs isolated from 10 ml of peripheral blood from newly diagnosed patients with BC, and paired intratumoral Treg cells (CD4⁺CD25⁺CD127⁻) (n=3) were sequenced. FIG. 8A shows the number of unique TCR nucleotide clone identified from intratumoral Treg cells and peripheral Treg cell subpopulations from each patient. FIGS. 8B-8C show the percentages of unique TCR nucleotide clonal overlap, *p<0.05 (FIG. 8B) and Morisita overlap index, *p<0.05 (FIG. 8C) between intratumoral Treg and Treg I, II or III cells, *p=0.046; **p=0.009. FIG. 8D shows the percentage of most frequent top 20 or top 50 intratumoral Treg TCR clones overlapped with Treg I, II or III cells, **p<0.005, One-way ANOVA. Shown are mean±s.e.m.

FIGS. 9A-9B demonstrates that intratumoral Treg cells have higher TCR clonal overlap with peripheral Treg II cells. (FIG. 9A) TCRβ CDR3 regions of flow sorted intratumor Treg cells (CD4⁺CD25⁺CD127⁻) and paired peripheral Treg I, II or III cells from patients with BC (n=3) were sequenced. Pair-wise scatter plots showing the overlapping TCR clones between intratumor Treg cells and peripheral Treg cell subpopulations. (FIG. 9B) The percentages of unique TCR nucleotide clonal overlap between peripheral blood Treg I, II or III cells. Shown are mean±s.e.m.

FIGS. 10A-10H demonstrate cytokine signaling responses in peripheral blood Treg II cells at diagnosis predicts future relapse of patients with BC. PBMCs from newly diagnosed patients with BC (n=40) were stimulated with TGFβ (25 ng/ml, 30 mins), IL-10 (100 ng/ml, 15 mins), IL-4 (50 ng/ml, 15 mins) or IFNγ (50 ng/ml, 15 mins). TGFβ-induced Smad2/3 phosphorylation, ****p<0.0001; *p=0.049 (FIG. 10A), IL-10-induced STAT1 phosphorylation, ****p<0.0001 (FIG. 10B), IL-4-induced STAT6 phosphorylation, ****p<0.0001 (FIG. 10C), or IFNγ-induced STAT1 phosphorylation, ***p=0.0007; *p=0.046 (FIG. 10D) in peripheral Tconv, Treg I, II and III cells were determined by phosphoflow cytometry with anti-p-Smad2 (pS465/pS467)/p-Smad3 (pS423/pS425), p-STAT1 (pY701), p-STAT6 (pY641) or p-STAT1 (pY701) antibodies, respectively. Signaling responses were quantified by cytokine-induced medium fluorescence intensity (MFI) minus the unstimulated MFI for p-Smad2/3 or p-STATs in Tconv, Treg I, II or III cell population. Friedman test. Shown are mean±s.e.m. FIGS. 10E-10H presents data using Kaplan-Meier estimate and log rank test, relapse-free survival (RFS) was compared between patients with BC (n=40) with low and high signaling response to TGFβ (FIG. 10E), IL-10 (FIG. 10F), IL-4 (FIG. 10G) or IFNγ (FIG. 10H) in peripheral blood Treg II cells. The median ΔMFI was used as the cut-off to divide patients with BC into low and high cytokine signaling response groups. All blood were collected from patients with BC at diagnosis before surgery or any therapy who had been clinically followed for at least 36 months.

FIGS. 11A-11D further demonstrate cytokine signaling responses in peripheral blood Treg II cells. FIG. 11A shows TGFβ-induced phosphorylation of Smad2/3 (p-Smad2/3) was higher in Treg II than in Tconv or Treg III cells. FIG. 1B shows IL-10-induced p-STAT1 was significantly higher in Treg II than in Tconv, Treg I or Treg III cells. Th2 cytokine IL-4-induced p-STAT6 and Th1 cytokine IFNγ-induced p-STAT1 (FIG. 11C) and Th cytokine IFNγ-induced p-STAT1 (FIG. 11D) were significantly lower in Treg II than in Tconv or Treg I cells.

FIGS. 12A-12J demonstrate Treg cell suppressive capacity could be reflected by cytokine signaling index (CSI). FIG. 12A shows Cytokine signaling index (CSI) of peripheral blood Treg cells is defined by the sum of z-score of TGFβ-induced p-Smad2/3 (ΔMFI), z-score of IL-10-induced p-STAT1 (ΔMFI), z-score of IL-4-induced p-STAT6 (ΔMFI) multiply by −1, and z-score of IFNγ-induced p-STAT1 (ΔMFI) multiply by −1. FIGS. 12B-12C presents data showing relapse-free survival (RFS) was compared between patients with breast cancer (BC) with above median and below median Treg II CSI from the discovery (n=40) (FIG. 12B) and validation cohort (n=38) (FIG. 12C) using Kaplan-Meier estimate and log rank test. All blood were collected from patients with BC at diagnosis before surgery or any therapy who had been clinically followed for at least 36 months. FIG. 12D is data showing Treg II CSI of patients with BC at diagnosis was compared between patients who remained relapse-free for at least 36 months (n=63) and patients who relapsed (n=15), ****p<0.0001, two-tailed, Mann Whitney test. Shown are mean±s.e.m. FIG. 12E is receiver operating characteristic (ROC) analysis for prognostic potential of Treg II CSI in patients with BC at diagnosis (n=78). FIG. 12F shows Treg II CSI of patients with BC at relapse was compared to patients with BC who are in remission for at least 36 months after most recent relapse. *p=0.041, two-tailed, Mann Whitney test. Shown are mean±s.e.m. FIG. 12G demonstrates the association between Treg II CSI and levels of CD39, CTLA4, OX40 in Treg II cells (n=40). FIGS. 12H-12J are data showing CellTrace Violet dilution by 104 labeled responder T cells (CD4⁺CD45RA⁺CD25⁻, Tresp) determined after 5 days of TCR-stimulated coculture with autologous peripheral Treg subpopulations from patients with BC at a 1 to 1 ratio (n=15). FIG. 12H is representative flow plots showing percentage of proliferated Tresp cells. FIG. 12I shows percent suppression was compared between Treg I, II and III cells. Treg cell suppression activity (suppression %) was calculated by: (1−% Tresp alone/% Tresp+Treg)×100%. ****p<0.0001; **p=0.007, Friedman test. Shown are mean±s.e.m. FIG. 12J reflects the association between Treg II CSI and percent suppression by Treg II cells. Spearman correlation coefficient test.

FIGS. 13A-13C presents data showing that cytokine signaling index of Tconv, Treg I or Treg III cells was not correlated with clinical outcome. Relapse-free survival (RFS) was compared between patients with BC (n=40) with above median CSI and below median CSI in peripheral Tconv (p=0.22) (FIG. 13A), Treg I (p=0.68) (FIG. 13B) or Treg III cells (p=0.07) (FIG. 13C) using Kaplan-Meier estimate and log rank test. All blood were collected from patients with BC at diagnosis before surgery or any therapy who had been clinically followed for at least 36 months. “ns”, not significant.

FIGS. 14A-14B present data investigating plasma levels of cytokines in patients with BC correlate with clinical outcome and/or reflect peripheral Treg II cell cytokine signaling response. Plasma levels of TGFβ, IL-10, IL-4 and IFNγ were similar between relapse-free and relapsed patients with BC (FIG. 14A), and no significant association between plasma levels of these cytokines with their signaling responses in peripheral Treg II cells (FIG. 14B).

FIGS. 15A-15C present data showing that Treg II CSI in healthy donors were lower than in relapsed patients with BC. (FIGS. 15A-15B) PBMCs from age-matched healthy donors (HD) (n=10) were stimulated with TGFβ (25 ng/ml, 30 mins), IL-10 (100 ng/ml, 15 mins), IL-4 (50 ng/ml, 15 mins) or IFNγ (50 ng/ml, 15 mins). TGFβ-induced p-Smad2/3 and IL-10-induced p-STAT1, ***p<0.001 (A), IFNγ-induced p-STAT1 and IL-4-induced p-STAT6 **p=0.008; *p=0.028 (FIG. 15B) in peripheral Treg II cells were determined by phosphoflow cytometry and were compared between healthy donors (n=10) and relapsed patients with BC (n=15). (FIG. 15C) Treg II CSI was compared between HD (n=10) and relapsed patients with BC (n=15), ****p<0.001, two-tailed, Mann-Whitney test. Shown are mean±s.e.m.

FIGS. 16A-16H demonstrate cytokine signaling index (CSI) of peripheral Treg II cells reflects immunosuppressive potential of intratumoral Treg cells. Multiplex immunofluorescence staining (FIG. 16A) and corresponding phenotype map (FIG. 16B) of representative untreated primary breast tumor tissue section for FoxP3 (green), CD8 (magenta), CD68 (cyan), CD123 (yellow), cytokeratin (CK, red) and DAPI (blue) Scale bar, 100 μm. FIG. 16C is a schematic representing the calculation of Treg interaction with other cell types based on the distribution of Treg cells within a radius of 20 μm from the nuclei of TAMs (CD68), CD8 T cells (CD8), pDCs (CD123) or cancer cells (CK). FIG. 16D shows comparison of percentage of Tregs within 20 μm from TAMs (Treg-TAM %) within the primary tumors from patients with BC who later relapsed (n=9) and from patients who remained relapse-free (n=11), *p=0.031, two-tailed, Mann Whitney test. Shown are mean±s.e.m. FIG. 16E presents data using Kaplan-Meier estimate and log rank test, relapse-free survival (RFS) was compared between patients with BC (n=20) with above median and below median % Treg-TAM in primary tumors. FIG. 16F shows the association between Treg-TAM % and the ratio of CD8/Treg (n=20). Spearman correlation coefficient test. FIG. 16G shows data where isolated peripheral blood monocytes (CD14+) from patients with BC were cocultured with flow sorted autologous peripheral II cells (2:1 ratio) for 5 days and levels of CCL2 were determined by ELISA (n=6), *p=0.03, two-tailed, Wilcoxon test. Shown are mean±s.e.m. FIG. 16H shows the association between Treg II CSI and % Treg-TAM (n=20). Spearman correlation coefficient test. *p<0.05.

FIGS. 17A-17D present data showing that cytokine signaling index (CSI) of peripheral Treg I or III cells not associated with immunosuppressive potential of intratumoral Treg cells. Untreated breast primary tumor tissue sections were stained for FoxP3, CD8, CD68, CD123, CK, and DAPI. (FIG. 17A) The density of Treg cells within primary tumors (FoxP3+ cell number per mm2) from patients with BC who later relapsed (n=9) and from patients who remained relapse-free (n=11), p=0.07, two-tailed, Mann-Whitney test, Shown are mean±s.e.m. (FIG. 17B) The association between Treg cell density and the percentage of Treg cells within 20 μm from TAMs (n=20) (p=0.14). (FIGS. 17C-17D) The association between Treg I CSI (p=0.52) (FIG. 17C) or Treg III CSI (p=0.15) (FIG. 17D) and the percentage of Treg cells within 20 μm from TAMs (n=20). Spearman correlation coefficient test. NS, not significant.

FIGS. 18A-18D demonstrate consistent cytokine signaling responses in reference PBMCs between discovery and validation runs. Different vials of reference PBMC from the same healthy donors (n=5) were thawed and run together with samples from patient with BC in the discovery and validation cohorts to determine consistency between runs. (FIG. 18A) TGF-induced Smad2/3 phosphorylation, (FIG. 18B) IL-10-induced STAT1 phosphorylation, (FIG. 18C) IL-4-induced STAT6 phosphorylation, or (FIG. 18D) IFNγ-induced STAT1 phosphorylation in T_{reg} II cells.

### DETAILED DESCRIPTION

### I. Definitions

Before the present invention is further described, it is to be understood that this invention is not strictly limited to particular embodiments described, as such may of course vary. It is also to be understood that the terminology used herein is for the purpose of describing particular embodiments only, and is not intended to be limiting, since the scope of the present invention will be limited only by the claims.

It must be noted that as used herein and in the appended claims, the singular forms “a,” “an,” and “the” include plural referents unless the context clearly dictates otherwise. It should further be understood that as used herein, the term “a” entity or “an” entity refers to one or more of that entity. For example, a nucleic acid molecule refers to one or more nucleic acid molecules. As such, the terms “a”, “an”, “one or more” and “at least one” can be used interchangeably. Similarly the terms “comprising”, “including” and “having” can be used interchangeably.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments are specifically embraced by the present invention and are disclosed herein just as if each and every combination was individually and explicitly disclosed. In addition, all sub-combinations are also specifically embraced by the present invention and are disclosed herein just as if each and every such sub-combination was individually and explicitly disclosed herein.

It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about means the specified value.

As used herein, the term “cancer” refers to all types of cancer, neoplasm or malignant tumors found in mammals (e.g. humans), including leukemias, lymphomas, carcinomas and sarcomas. Exemplary cancers that may be treated with a compound or method provided herein include brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer. In embodiments, the cancer is melanoma. In embodiments, the cancer is gastrointestinal cancer. In embodiments, the cancer is breast cancer.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human. In embodiments, the subject has, had, or is suspected of having cancer.

“Breast cancer patient” or “breast cancer subject” refers to a patient or subject with breast cancer. In embodiments, the breast cancer patient or breast cancer subject has, had, or is suspected of having breast cancer. In embodiments, the breast cancer patient or breast cancer subject has breast cancer. In embodiments, the breast cancer patient or breast cancer subject had breast cancer. In embodiments, the breast cancer patient or breast cancer subject is suspected of having breast cancer. In embodiments, the breast cancer patient or breast cancer subject is at risk of having a relapse of breast cancer.

“Breast cancer” refers to a malignant tumor that develops from cells in the breast. Types of breast cancer include ductal carcinoma in situ, invasive ductal carcinoma, tubular carcinoma of the breast, medullary carcinoma of the breast, mucinous carcinoma of the breast, papillary carcinoma of the breast, cribriform carcinoma of the breast, invasive lobular carcinoma, inflammatory breast cancer, lobular carcinoma in situ, and the like. The breast cancer may also be of a molecular sub-type, such as luminal A, luminal B, triple negative, HER2, and normal-like. The breast cancer can be primary breast cancer or metastatic breast cancer. The breast cancer can be any stage, including Stage 0, IA, IB, IIA, IIIB, IIIA, IIIB, IIIC, or IV.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

As used herein, the terms “treating” or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

As used herein, the term “prevent” refers to a decrease in the occurrence of disease symptoms in a patient. As indicated above, the prevention may be complete (no detectable symptoms) or partial, such that fewer symptoms are observed than would likely occur absent treatment.

As used herein, the term “relapse” refers to the clinical diagnosis of a return of cancer after a period of remission.

As used herein, the term “relapse-free survival” or “RFS” refers to the time from the date of diagnosis of cancer to the date of relapse.

As used herein, the term “biological sample” refers to a material of biological origin (e.g., blood, plasma, cells, tissues, organs, fluids). In embodiments, “biological sample” is a blood sample. In embodiments, the “blood sample” includes blood cells. In embodiments, blood cells are isolated from the blood sample, thereby creating an isolated blood cell fraction that includes isolated blood sample cells. In embodiments, the isolated blood sample cells are leukocytes.

As used herein, the term “peripheral blood” refers to blood circulating throughout the body. The components of peripheral blood include red blood cells (erythrocytes), white blood cells (leukocytes), and platelets.

As used herein, the term “peripheral blood mononuclear cell” or “PBMC” refers to cells in peripheral blood that have a nucleus, generally a round nucleus. Exemplary peripheral blood mononuclear cells include lymphocytes and monocytes. Exemplary lymphocytes are T cells, B cells, and NK cells. Peripheral blood mononuclear cells can be extracted from blood (e.g., peripheral blood) by methods known in the art.

As used herein, the term “immune response” and the like refer, in the usual and customary sense, to a response by an organism that protects against disease. The response can be mounted by the innate immune system or by the adaptive immune system, as well known in the art.

As used herein, the term “modulating immune response” and the like refer to a change in the immune response of a subject as a consequence of administration of an agent, e.g., a compound as disclosed herein, including embodiments thereof. Accordingly, an immune response can be activated or deactivated as a consequence of administration of an agent, e.g., a compound as disclosed herein, including embodiments thereof.

As used herein, the term “T cells” or “T lymphocytes” are a type of lymphocyte (a subtype of white blood cell) that plays a central role in cell-mediated immunity. They can be distinguished from other lymphocytes, such as B cells and natural killer cells, by the presence of a T-cell receptor on the cell surface. T cells include, for example, natural killer T (NKT) cells, cytotoxic T lymphocytes (CTLs), regulatory T (Treg) cells, and T helper cells. Different types of T cells can be distinguished by use of T cell detection agents.

As used herein, the term “memory T cell” refers to a T cell that has previously encountered and responded to its cognate antigen during prior infection, encounter with cancer or previous vaccination. At a second encounter with its cognate antigen memory T cells can reproduce (divide) to mount a faster and stronger immune response than the first time the immune system responded to the pathogen.

As used herein, the term “regulatory T cell” or “suppressor T cell” refers to a lymphocyte which modulates the immune system, maintains tolerance to self-antigens, and prevents autoimmune disease.

As used herein, the term “monocytes” refers to type of leukocyte. They are the largest type of leukocyte and can differentiate into macrophages and myeloid lineage dendritic cells. As a part of the vertebrate innate immune system monocytes also influence the process of adaptive immunity. There are at least three subclasses of monocytes in human blood based on their phenotypic receptors, specifically CD14 and CD16.

As used herein, the term “tumor microenvironment” or “cancer microenvironment” refers to the non-neoplastic cellular environment of a tumor, including blood vessels, immune cells, fibroblasts, cytokines, chemokines, non-cancerous cells present in the tumor, and proteins produced

As defined herein, the term “activation”, “activate”, “activating”, “activator” and the like in reference to a protein-inhibitor interaction means positively affecting (e.g. increasing) the activity or function of the protein relative to the activity or function of the protein in the absence of the activator. In embodiments activation means positively affecting (e.g. increasing) the concentration or levels of the protein relative to the concentration or level of the protein in the absence of the activator. The terms may reference activation, or activating, sensitizing, or up-regulating signal transduction or enzymatic activity or the amount of a protein decreased in a disease. Thus, activation may include, at least in part, partially or totally increasing stimulation, increasing or enabling activation, or activating, sensitizing, or up-regulating signal transduction or enzymatic activity or the amount of a protein associated with a disease (e.g., a protein which is decreased in a disease relative to a non-diseased control). Activation may include, at least in part, partially or totally increasing stimulation, increasing or enabling activation, or activating, sensitizing, or up-regulating signal transduction or enzymatic activity or the amount of a protein

As used herein, the terms “agonist,” “activator,” “upregulator,” etc. refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist.

As used herein, the terms “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. In embodiments inhibition means negatively affecting (e.g. decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein or cell from an indirect interaction (e.g. an inhibitor binds to a protein that activates the target protein, thereby preventing target protein activation or cell activations).

As used herein, the terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

As used herein, the term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

As used herein, the term “modulator” refers to a composition that increases or decreases the level of a target molecule or the function of a target molecule or the physical state of the target of the molecule relative to the absence of the modulator.

As used herein, the term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

As used herein, the term “associated” or “associated with” in the context of a substance or substance activity or function associated with a disease (e.g. a protein associated disease, a cancer (e.g., cancer, inflammatory disease, autoimmune disease, or infectious disease)) means that the disease (e.g. cancer, inflammatory disease, autoimmune disease, or infectious disease) is caused by (in whole or in part), or a symptom of the disease is caused by (in whole or in part) the substance or substance activity or function. As used herein, what is described as being associated with a disease, if a causative agent, could be a target for treatment of the disease.

As used herein, the term “signaling pathway” refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components.

As used herein, the term “CD45RA” refers to the CD45 receptor antigen also known as Protein tyrosine phosphatase, receptor type, C (PTPRC). Exemplary amino acid sequences for CD45RA include GENBANK® Accession Nos. NP_002829.3, NP_563578.2, NP_563578.2, and NP_002829.3.

As used herein, the terms “FOXP3” or “forkhead box P3”, also known as “scurfin” are used herein according to its plain and ordinary meaning and refer to a protein involved in immune system responses. A member of the FOX protein family, FOXP3 may function as a master regulator of the regulatory pathway in the development and function of regulatory T cells. Regulatory T cells generally turn the immune response down. In cancer, an excess of regulatory T cell activity may prevent the immune system from destroying cancer cells. In autoimmune disease, a deficiency of regulatory T cell activity can allow other autoimmune cells to attack the body's own tissue

As used herein, the term “cytokine” refers to a broad category of small proteins (˜5-20 kDa) that are important in cell signaling. Cytokines are peptides, and cannot cross the lipid bilayer of cells to enter the cytoplasm. Cytokines are involved in autocrine signaling, paracrine signaling and endocrine signaling as immunomodulating agents. Cytokines include chemokines, interferons, interleukins, lymphokines, and tumor necrosis factors. Cytokines are produced by a broad range of cells, including immune cells like macrophages, B lymphocytes, T lymphocytes and mast cells, as well as endothelial cells, fibroblasts, and various stromal cells; a given cytokine may be produced by more than one type of cell.

For specific proteins described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

As used herein, the term “CCR8” is used herein according to its plain and ordinary meaning and refers to a member of the beta chemokine receptor family. The ligand of CCR8 is CCL1. It has a role in regulation of monocyte chemotaxis and thymic cell apoptosis. Also included are variants or homologs thereof that maintain CCR8 activity (e.g. within at least 50%, 80%, 90%, 95%, 96%, 97%, 98%, 99% or 100% activity compared to CCR8). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CCR8.

As used herein, the term “CCL1” is used herein according to its plain and ordinary meaning and refers to a small glycoprotein secreted by activated T cells that belongs to a family of inflammatory cytokines known as chemokines. CCL1 attracts monocytes, NK cells, and immature B cells and dendritic cells by interacting with a cell surface chemokine receptor called CCR8.

As used herein, the terms “T cell receptor” and “TCR” is a molecule found on the surface of T cells, or T lymphocytes, that is responsible for recognizing fragments of antigen as peptides bound to major histocompatibility complex (MHC) molecules. The binding between TCR and antigen peptides is of relatively low affinity and is degenerate: that is, many TCRs recognize the same antigen peptide and many antigen peptides are recognized by the same TCR. The TCR is composed of two different protein chains (that is, it is a heterodimer). In humans, in 95% of T cells the TCR consists of an alpha (α) chain and a beta (β) chain, whereas in 5% of T cells the TCR consists of gamma and delta (γ/δ) chains (encoded by TRG and TRD, respectively).

As used herein, the terms “TGF-beta” or “TGF-β” are used herein according to its plain and ordinary meaning and refer to a multifunctional cytokine belonging to the transforming growth factor superfamily. TGF-β proteins are produced by all white blood cell lineages.

As used herein, the term “IL-4” is used herein according to its plain and ordinary meaning and refers to any of the recombinant or naturally-occurring forms of IL-4 or variants or homologs thereof that maintain IL-4 activity (e.g. within at least 50%, 80%, 90%, 95%, 96%, 97%, 98%, 99% or 100% activity compared to IL-4). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IL-4. IL-4 is a cytokine that induces differentiation of naive helper T cells (Th0 cells) to Th2 cells. Upon activation by IL-4, Th2 cells subsequently produce additional IL-4 in a positive feedback loop. The cell that initially produces IL-4, thus inducing Th2 differentiation, has not been identified, but recent studies suggest that basophils may be the effector cell. It is closely related and has functions similar to interleukin 13. Interleukin 4 has many biological roles, including the stimulation of activated B-cell and T-cell proliferation, and the differentiation of B cells into plasma cells. It is a key regulator in humoral and adaptive immunity. IL-4 induces B-cell class switching to IgE, and up-regulates MHC class II production. IL-4 decreases the production of Th1 cells, macrophages, IFN-gamma, and dendritic cell IL-12.

As used herein, the terms “IL-10”, “Interleukin 10”, and “human cytokine synthesis inhibitory factor (CSIF)” are used herein according to their plain and ordinary meaning and refer to any of the recombinant or naturally-occurring forms of the anti-inflammatory cytokine Interleukin-10 (IL-10) or variants or homologs thereof that maintain IL-10 activity (e.g. within at least 50%, 80%, 90%, 95%, 96%, 97%, 98%, 99% or 100% activity compared to IL-10). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IL-10. IL-10 signals through a receptor complex consisting of two IL-10 receptor-1 and two IL-10 receptor-2 proteins. IL-10 is a cytokine with multiple, pleiotropic, effects in immunoregulation and inflammation. It downregulates the expression of Th1 cytokines, MHC class II antigens, and co-stimulatory molecules on macrophages. It also enhances B cell survival, proliferation, and antibody production. IL-10 can block NF-κB activity, and is involved in the regulation of the JAK-STAT signaling pathway.

As used herein, the terms “IFN-γ” and “interferon gamma” are used herein according to its plain and ordinary meaning and refer to a dimerized soluble cytokine that is the only member of the type II class of interferons. It plays a role in innate and adaptive immunity against viral, some bacterial and protozoal infections. IFNγ is an important activator of macrophages and inducer of Class II major histocompatibility complex (MHC) molecule expression. The importance of IFNγ in the immune system stems in part from its ability to inhibit viral replication directly, and most importantly from its immunostimulatory and immunomodulatory effects. IFNγ is produced predominantly by natural killer (NK) and natural killer T (NKT) cells as part of the innate immune response, and by CD4 Th1 and CD8 cytotoxic T lymphocyte (CTL) effector T cells once antigen-specific immunity develops.

As used herein, the terms “CTLA-4” or “CTLA-4 protein” are used herein according to its plain and ordinary meaning and refer to any of the recombinant or naturally-occurring forms of the cytotoxic T-lymphocyte-associated protein 4 (CTLA-4) or variants or homologs thereof that maintain CTLA-4 protein activity (e.g. within at least 50%, 80%, 90%, 95%, 96%, 97%, 98%, 99% or 100% activity compared to CTLA-4). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CTLA-4 polypeptide. In embodiments, CTLA-4 is the protein as identified by the NCBI sequence reference GI:83700231, homolog or functional fragment thereof.

As used herein, the term “OX40” or “OX40 protein” are used herein according to its plain and ordinary meaning and refer to any of the recombinant or naturally-occurring forms of tumor necrosis factor receptor superfamily, member 4 (OX40) also known as cluster of differentiation 134 (CD 134) or variants or homologs thereof that maintain OX40 activity (e.g. within at least 50%, 80%, 90%, 95%, 96%, 97%, 98%, 99% or 100% activity compared to OX40). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring OX40 protein. In embodiments, the OX40 protein is substantially identical to the protein identified by the UniProt reference number P43489 or a variant or homolog having substantial identity thereto.

As used herein, the term “anticancer agent” refers to a molecule or composition (e.g. compound, peptide, protein, nucleic acid, drug, antagonist, inhibitor, modulator) used to treat cancer through destruction or inhibition of cancer cells or tissues. Anticancer agents may be selective for certain cancers or certain tissues. In some embodiments, an anti-cancer agent is a chemotherapeutic. In embodiments, anticancer agents herein may include epigenetic inhibitors and multi-kinase inhibitors.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### II. Methods of Use

In an aspect, provided herein are methods of detecting cytokine signaling responsiveness in immune cells from a cancer subject. The methods include isolating cells from a blood sample from the cancer subject thereby forming isolated blood cell fraction that includes isolated blood sample cells, mixing the isolated blood sample cells with a cytokine, where the cytokine is selected from TGFβ, IL-10, IL-4 and IFNγ, and detecting the responsiveness of the isolated blood sample cells to the cytokine.

In an aspect, provided herein are methods of determining risk of relapse of cancer in a subject. The methods include isolating cells from a blood sample from the subject thereby forming an isolated blood cell fraction that includes isolated blood sample cells, mixing the isolated blood sample cells with a cytokine, where the cytokine is selected from TGFβ, IL-10, IL-4 or IFNγ, detecting the responsiveness of the isolated blood sample cells to the cytokine, and determining whether said subject has a high risk of cancer relapse.

In an aspect, provided herein are methods of treating a cancer in a subject in need thereof. The methods include isolating cells from a blood sample from the subject thereby forming an isolated blood cell fraction comprising isolated blood sample cells, mixing the isolated blood sample cells with a cytokine, where the cytokine is selected from TGFβ, IL-10, IL-4 and IFNγ, detecting the responsiveness of the isolated blood sample cells to the cytokine, and treating said subject with a therapeutic regimen.

In embodiments, the cancer subject currently has, has been diagnosed with cancer, previously had, or is suspected of having cancer. In embodiments, the cancer subject currently has cancer. In embodiments, the cancer subject has been diagnosed with cancer. In embodiments, the cancer subject previously had cancer. In embodiments, the cancer subject is suspected of having cancer.

In embodiments, the cancer is selected from breast, melanoma, or gastrointestinal cancer. In embodiments, the cancer is breast cancer. In embodiments, the cancer is melanoma. In embodiments, the cancer is gastrointestinal.

In embodiments, the cancer subject currently has, has been diagnosed with, previously had, or is suspected of having breast cancer. In embodiments, the cancer subject currently has breast cancer. In embodiments, the cancer subject has been diagnosed with breast cancer. In embodiments, the cancer subject previously had breast cancer. In embodiments, the cancer subject is suspected of having breast cancer.

In embodiments, the methods include isolating cells from a blood sample from the subject thereby forming an isolated blood cell fraction that includes isolated blood sample cells. In embodiments, the method of isolating blood cells includes density centrifugation, FACS (fluorescence activated cell sorting), MACS (magnetically activated cell sorting), aptamer binding, and the like. In embodiments, the method of isolating blood cells is density centrifugation. In embodiments, the method of isolating blood cells is Ficoll-Paque density centrifugation. In embodiments, the method of isolating blood cells is FACS (fluorescence activated cell sorting).

In embodiments, the isolated blood sample cells are leukocytes. In embodiments, the isolated blood sample cells are leukocytes selected from lymphocytes and monocytes. In embodiments, the leukocyte is a monocyte. In embodiments, the leukocyte is a lymphocyte. In embodiments, the lymphocyte is a T-cell. In embodiments, the T-cell is a regulatory T-cell. In embodiments, the regulatory T-cell is selected from Treg I, Treg II, and Treg III type cell. In embodiments, the regulatory T-cell is a Treg II cell. In embodiments, the Treg II cell is CD4⁺CD45RA⁻FoxP3^{hi}. In embodiments, the Treg II cell is CD45RA⁻FoxP3^{hi}.

In embodiments, the method includes mixing the isolated blood sample cells with a cytokine. In embodiments, the method includes mixing the isolated blood sample cells with one or more cytokines. In embodiments, the method includes mixing the isolated blood sample cells with two or more cytokines. In embodiments, the method includes mixing the isolated blood sample cells with three or more cytokines. In embodiments, the method includes mixing the isolated blood sample cells with four or more cytokines. In embodiments, the method includes mixing the isolated blood sample cells with one cytokine. In embodiments, the method includes mixing the isolated blood sample cells with two cytokines. In embodiments, the method includes mixing the isolated blood sample cells with three cytokines. In embodiments, the method includes mixing the isolated blood sample cells with four cytokines.

In embodiments, the cytokine is selected from TGFβ, IL-10, IL-4 and IFNγ. In embodiments, the cytokine is TGFβ. In embodiments, the cytokine is IL-10. In embodiments, the cytokine is IL-4. In embodiments, the cytokine is IFNγ.

In embodiments, the method includes mixing the isolated blood sample cells with two or more cytokines. In embodiments, the two or more cytokines are selected from TGFβ, IL-10, IL-4 and IFNγ. In embodiments, the two or more cytokines are TGFβ and IL-10. In embodiments, the two or more cytokines are TGFβ and IL-10. In embodiments, the two or more cytokines are TGFβ and IFNγ. In embodiments, the two or more cytokines are IL-10 and IL-4. In embodiments, the two or more cytokines are IL-10 and IFNγ. In embodiments, the two or more cytokines are IL-4 and IFNγ.

In embodiments, the method includes mixing the isolated blood sample cells with three or more cytokines. In embodiments, the three or more cytokines are selected from TGFβ, IL-10, IL-4 and IFNγ. In embodiments, the three or more cytokines are TGFβ, IL-10, and IL-4. In embodiments, the three or more cytokines are TGFβ, IL-4, and IFNγ. In embodiments, the three or more cytokines are TGFβ, IL-10, and IFNγ. n embodiments, the three or more cytokines are IL-10, IL-4 and IFNγ.

In embodiments, the method further includes mixing the isolated blood sample cells with a cytokine selected from IL-2, IL-3, IL-5, IL-6, IL-7, IL-8, IL-9, IL-11, IL-12, IL-13, IL-15, C-NTF, LIF, OSM (Oncostatin-M), EPO (Erythropoietin), G-CSF (GCSF), GH (Growth Hormone), PRL (Prolactin), IFN-alpha, IFN-beta, GM-CSF, M-CSF, SCF, IL1-alpha, IL1-beta, aFGF (FGF-acidic), bFGF (FGF-basic), INT-2, KGF (FGF7), EGF, TGF-alpha, Betacellulin (BTC), SCDGF, Amphiregulin, TNF-beta, PDGF, and HB-EGF.

In embodiments, the method further includes mixing the isolated blood sample cells with one or more, two or more, three or more, four or more cytokine selected from IL-2, IL-3, IL-5, IL-6, IL-7, IL-8, IL-9, IL-11, IL-12, IL-13, IL-15, C-NTF, LIF, OSM (Oncostatin-M), EPO (Erythropoietin), G-CSF (GCSF), GH (Growth Hormone), PRL (Prolactin), IFN-alpha, IFN-beta, GM-CSF, M-CSF, SCF, IL1-alpha, IL1-beta, aFGF (FGF-acidic), bFGF (FGF-basic), INT-2, KGF (FGF7), EGF, TGF-alpha, Betacellulin (BTC), SCDGF, Amphiregulin, TNF-beta, PDGF, and HB-EGF.

In embodiments, the method further includes mixing the isolated blood sample cells with 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, or 38 cytokines selected from IL-2, IL-3, IL-5, IL-6, IL-7, IL-8, IL-9, IL-11, IL-12, IL-13, IL-15, C-NTF, LIF, OSM (Oncostatin-M), EPO (Erythropoietin), G-CSF (GCSF), GH (Growth Hormone), PRL (Prolactin), IFN-alpha, IFN-beta, GM-CSF, M-CSF, SCF, IL1-alpha, IL1-beta, aFGF (FGF-acidic), bFGF (FGF-basic), INT-2, KGF (FGF7), EGF, TGF-alpha, Betacellulin (BTC), SCDGF, Amphiregulin, TNF-beta, PDGF, and HB-EGF.

In embodiments, the method includes detecting the responsiveness of the isolated blood sample cells to a cytokine. Cytokines interact with specific high-affinity cell surface receptors, which is followed by a cascade of signal transduction events, resulting in mRNA synthesis and, eventually, protein secretion. Detecting the responsiveness of a cell to a cytokine is intended to mean a process of detecting a change in a cell parameter resulting from the interaction between the cell and a cytokine. Cell parameters may include a phenotype change, a change in gene expression, a change in cell metabolism or change in a cell signal transduction (e.g. phosphorylation of a protein). In embodiments, detecting responsiveness includes measuring cytokine-induced phosphorylation of a target transcription factor. In embodiments, measuring phosphorylation of a target transcription factor includes phosphoflow cytometry.

Immune signaling responses may be considered relative. In cancer patients, signaling to some cytokines go up, some go down, and many stay the same. Embodiments herein provide methods for detecting signaling to multiple cytokines and creating a formula to calculate a weighted score that also accounts for the different directionality of changes of any individual cytokine compared to a standard control. In embodiments, methods provided herein include methods for detecting responsiveness of immune cells in a blood sample to one or more cytokines, quantifying the responsiveness to calculate a cytokine signaling responsive index, and determining a risk of cancer relapse in a subject.

In embodiments, detecting includes quantifying an amount of responsiveness of the isolated blood sample cells to a cytokine. In embodiments, the isolated blood sample cells include immune cells. In embodiments, quantifying includes calculating a cytokine signaling index. In embodiments, a cytokine signaling index is used to determine whether said subject is at risk of cancer relapse.

In embodiments, quantifying the responsiveness of isolated blood sample cells to one or more cytokines includes comparing the amount of responsiveness to a standard control, where if the amount is higher than the standard control, said cancer subject has a high risk of cancer relapse. In embodiments, quantifying the responsiveness of isolated blood sample cells to one or more cytokines includes comparing the amount to a standard control, where if the amount is lower than the standard control, said cancer subject has a high risk of cancer relapse. In embodiments, the isolated blood sample cells include immune cells.

In embodiments, quantifying an amount of responsiveness of the isolated blood sample cells to a cytokine includes calculating a cytokine signaling responsive index (may also be referred to as a cytokine signaling index or CSI). In embodiments, calculating a cytokine signaling responsive index includes mathematical transformation of the values of detection of responsiveness to each cytokine. In embodiments, the calculation takes into account all of these different weighted directional changes in high risk patients. In embodiments, the methods provided herein relate cytokine signaling responsiveness in immune cells from peripheral blood to cancer relapse risk.

In embodiments, determining a risk of cancer relapse includes comparing a calculated signaling responsive index to a standard control. In embodiments, if the index is greater than the standard control, the risk of cancer relapse is high. In embodiments, if the index is less than the standard control, the risk of cancer relapse is high. In embodiments, the standard control is the CSI median.

In embodiments, the standard control is derived from a reference group. In embodiments, the reference group is age matched subjects who do not have cancer. In embodiments, the standard control is derived from a reference group. In embodiments, the reference group is sex matched subjects who do not have cancer. In embodiments, the standard control is derived from a reference group. In embodiments, the reference group is age and sex matched subjects who do not have cancer. In embodiments, for breast cancer, the reference group is healthy women. In embodiments, for breast cancer, the reference group is women without breast cancer. For other cancers such as melanoma or gastrointestinal cancer, the reference group includes healthy or cancer free individuals from both sexes.

In embodiments, CSI is calculated by using the sum of Z-score of TGFβ-induced pSmad2/3, IL-10-induced pSTAT1 and Z-score of IL-4-induced pSTAT6 and IFNγ-induced pSTAT1 multiplied by −1 in Treg II from patients with breast cancer (discovery and validation cohort combined). In embodiments, the CSI is calculated based on the cohort of patients used. In embodiments, the CSI median is used to divide the patient cohorts.

In embodiments, the methods include an intervention if the risk of cancer relapse is high in the subject. In embodiments, the intervention includes more closely monitoring for cancer relapse, selecting the patient for more aggressive therapy, or a combination thereof. In embodiments, the intervention includes more closely monitoring for cancer relapse. In embodiments, closer monitoring includes great frequency of checkups, additional diagnostic interventions, and/or a combination thereof. In embodiments, the intervention includes selecting the patient for more aggressive therapy. In embodiments, aggressive therapy includes higher dosage, greater frequency, experimental therapeutics, and/or a combination thereof. The therapeutics may include chemotherapy, radiation therapy, and/or immunotherapy. In embodiments, the intervention includes more closely monitoring for cancer relapse and selecting the patient for more aggressive therapy.

In embodiments, the methods include an aggressive therapeutic regimen including one or more of a chemotherapy combination of cyclophosphamide, methotrexate, fluorouracil, adriamycin, and taxane. In embodiments, the chemotherapy combination is cyclophosphamide, methotrexate, and fluorouracil. In embodiments, the chemotherapy combination is cyclophosphamide, adriamycin, and fluorouracil. In embodiments, the chemotherapy combination is adriamycin and cyclophosphamide. In embodiments, the chemotherapy combination is adriamycin, cyclophosphamide, and taxane. In embodiments, the chemotherapy combination is fluorouracil, adriamycin, and cyclophosphamide. In embodiments, the chemotherapy combination is fluorouracil, adriamycin, cyclophosphamide, and taxane.

In an aspect, provided herein is a method of preparing a sample including isolating cells from a blood sample from a subject thereby forming an isolated blood cell fraction including isolated blood sample cells, mixing the isolated blood sample cells with a cytokine, wherein said cytokine is selected from TGFβ, IL-10, IL-4 and IFNγ, detecting the responsiveness of the isolated blood sample cells to said cytokine, and quantifying an amount of responsiveness of the isolated blood sample cells to said cytokine.

### Examples

### Example 1: Connecting Blood and Intratumoral Treg Cell Activity in Predicting Future Relapse in Breast Cancer

The experiments described herein demonstrate that the peripheral blood CD45RA⁻FoxP3^{hi} Treg II cells are phenotypically closest to intratumoral Treg cells, including CCR8 expression. T cell receptor repertoire analyses further support that intratumoral Treg cells may originate primarily from peripheral blood Treg II cells. Moreover, signaling responsiveness to immunosuppressive, Th1 and Th2 cytokines in peripheral blood Treg II cells at diagnosis reflects intratumoral immunosuppressive potential and predicts future relapse in two independent cohorts of patients with breast cancer. Together, these findings shed important insights into the relationship between peripheral blood Treg with intratumoral Treg cells, and highlight cytokine signaling responsiveness as key determinants of intratumoral immunosuppressive potential and clinical outcome.

Treg cells are heterogeneous in phenotype and function, with three distinct subpopulations identified within human peripheral blood: CD45RA⁺FoxP3^{lo} (Treg I), CD45RA⁻FoxP3^{hi} (Treg II) and CD45RA⁻FoxP3^{lo} (Treg III) (see for example, Ref. 14). The frequency of peripheral blood Treg cells is elevated in patients with breast cancer (BC), and is reported to correlate with clinical response to anti-CTLA4 treatment and adoptive cellular immunotherapy (see for example, 15-17). However, how the functional activity of peripheral blood Treg cells relates to immunosuppressive activity of intratumoral Treg cells and whether different peripheral Treg cell subpopulations have different tumor infiltration potential remain unclear. Accordingly, a blood-based assay of peripheral blood Tregs that reflects immunosuppressive potential of intratumoral Treg cells from cancer patients would be particularly useful.

The experiments described herein sought to understand the relationship between Treg cell subpopulations in peripheral blood with intratumoral Treg cells via detailed characterization of paired samples from patients with BC, including major immune-regulating proteins, chemokine expression patterns, and T cell receptor (TCR) repertoire analysis. The clinical significance of signaling responsiveness to key immunosuppressive and immunostimulatory cytokines in peripheral blood Treg cells from newly diagnosed patients with BC was analyzed. Signaling responsiveness to individual cytokines was combined into a cytokine signaling index (CSI) to derive an integrated understanding of their immunosuppressive potential in relation to clinical outcome in patients with BC.

Results

Immune Phenotype of Peripheral Blood Treg II Cells is Similar to Paired Intratumoral Treg Cells.

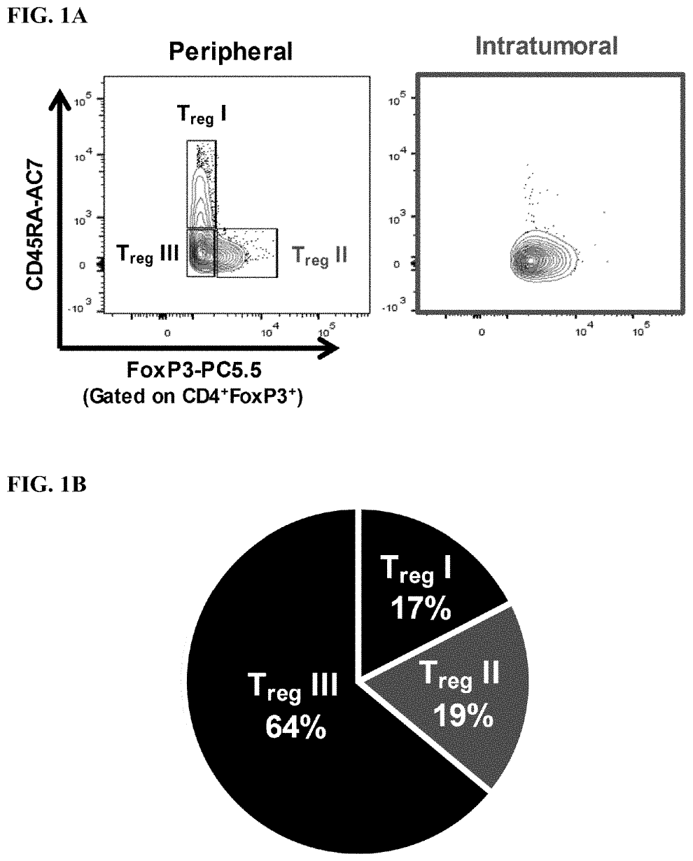

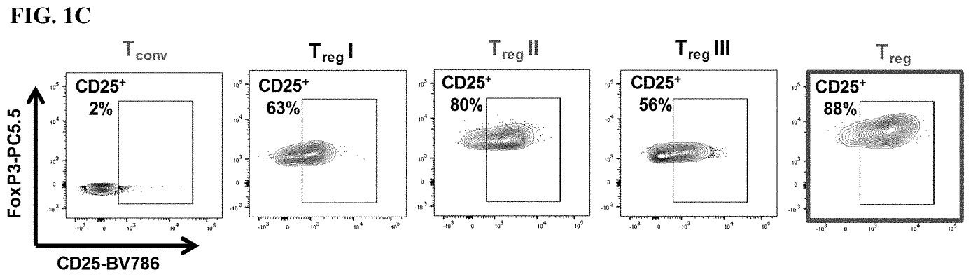

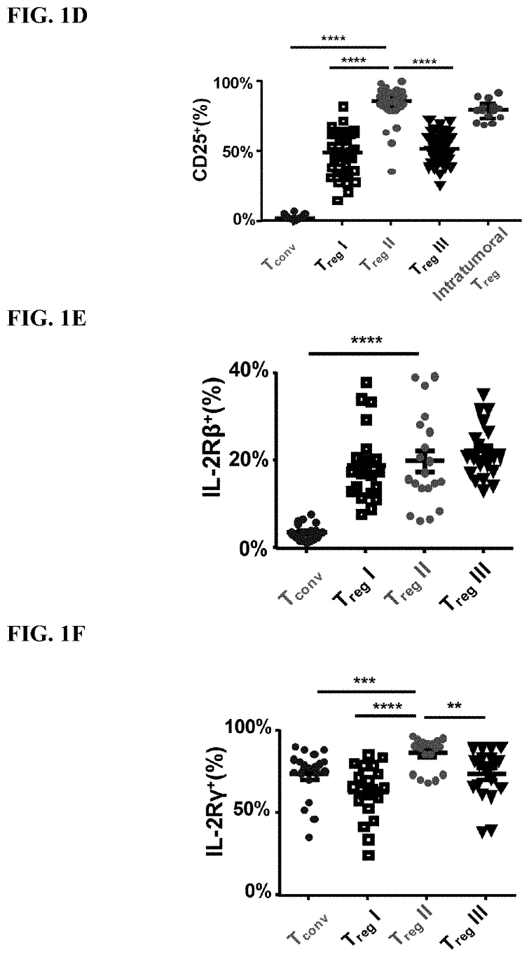

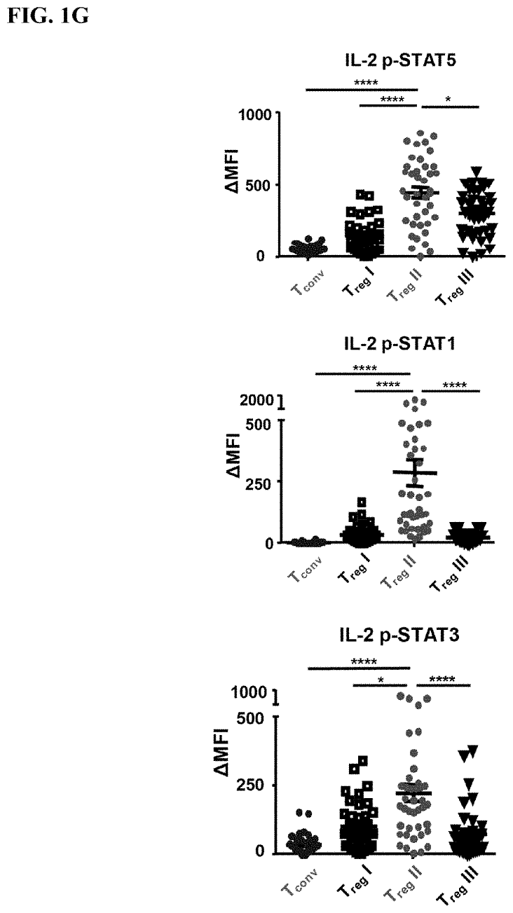

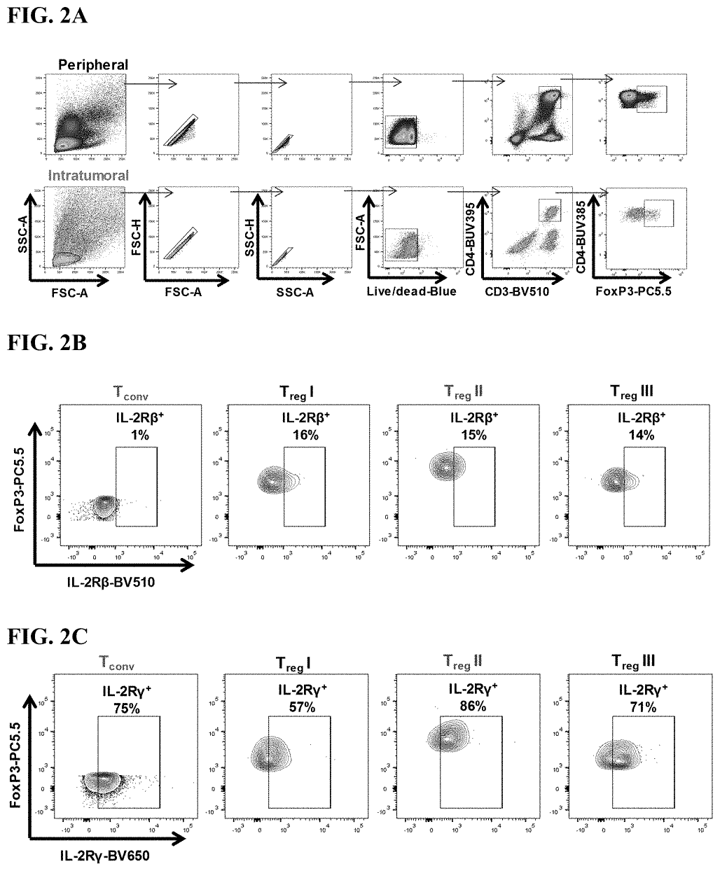

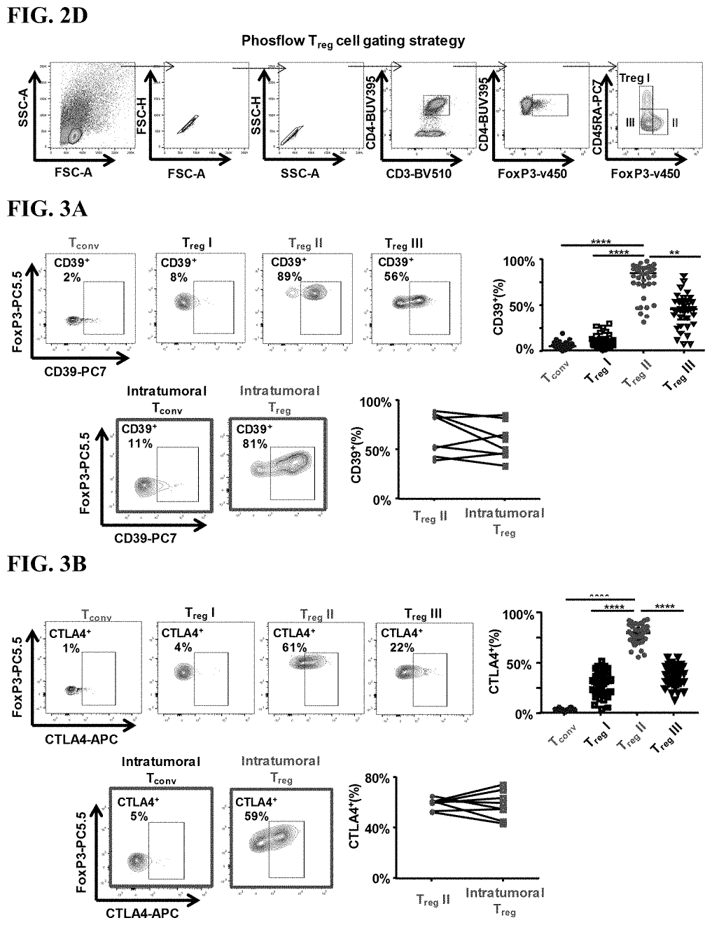

Peripheral blood Treg cells (CD4⁺FoxP3⁺) from patients with BC can be clearly separated into three distinct subpopulations consistent with findings from healthy subjects: CD45RA⁺ FoxP3^{lo} Treg I cells, CD45RA⁻FoxP3^{hi} Treg II cells, and CD45RA⁻FoxP3^{lo} Treg III cells (FIG. 1A) (gating strategy in FIG. 2A). The average compositions of peripheral blood FoxP3+ Treg cell subpopulations from untreated patients with BC at diagnosis (n=118) were 17% Treg I, 19% Treg II, and 64% Treg III cells (FIG. 1B). In contrast, nearly all intratumoral Treg cells from human primary breast tumors (n=28) were CD45RA⁻FoxP3^{+/hi}, similar to Treg II cells (FIG. 1A). Since IL-2 competition/consumption is one of the well-known immunosuppressive mechanisms of Treg cells and IL-2-induced signal transducer and activator of transcription (STAT) 5 activation is essential for Treg cell function (see for example Ref. 18), levels of the three subunits of IL-2 receptor complex on peripheral blood Treg cell subpopulations were compared against conventional CD4+ T cells (Tconv cell, CD4⁺FoxP3⁻). Data demonstrated that the percentage of CD25⁺ was significantly higher in Treg II cells than in Tconv, Treg I, or Treg III cells and CD25 was also highly expressed in paired intratumoral Tregs (FIG. 1C-D). Notably, the percentage of IL-2Rβ+ was similar amongst all Treg subpopulations, and higher than in Tconv (FIG. 1E) (representative flow plots in FIG. 2B). Lastly, levels of IL-2Rγ were also significantly higher on Treg II cells than on Tconv, Treg I or III cells (FIG. 1F) (representative flow plots in FIG. 2C).

To investigate IL-2-induced signaling responses in peripheral blood Treg subpopulations, IL-2-induced phosphorylation of STAT5 (pSTAT5), which is the major IL-2-induced downstream signaling molecule for FoxP3 expression and Treg cell differentiation was measured (see for example Ref. 19). IL-2-induced STAT1 and STAT3 phosphorylation (p-STAT1/3) in peripheral Treg cell subpopulations (n=40) was analyzed (gating strategy in FIG. 2D). As expected, IL-2-induced pSTAT5 was significantly higher in Treg II cells than in Tconv, Treg I or Treg III cells (FIG. 1G). Intriguingly, IL-2 also induced higher p-STAT1 and pSTAT3 (FIG. 1G) in Treg II cells than in Tconv, Treg I or Treg III cells, indicating that IL-2 may induce more diverse downstream signaling in Treg II cells.

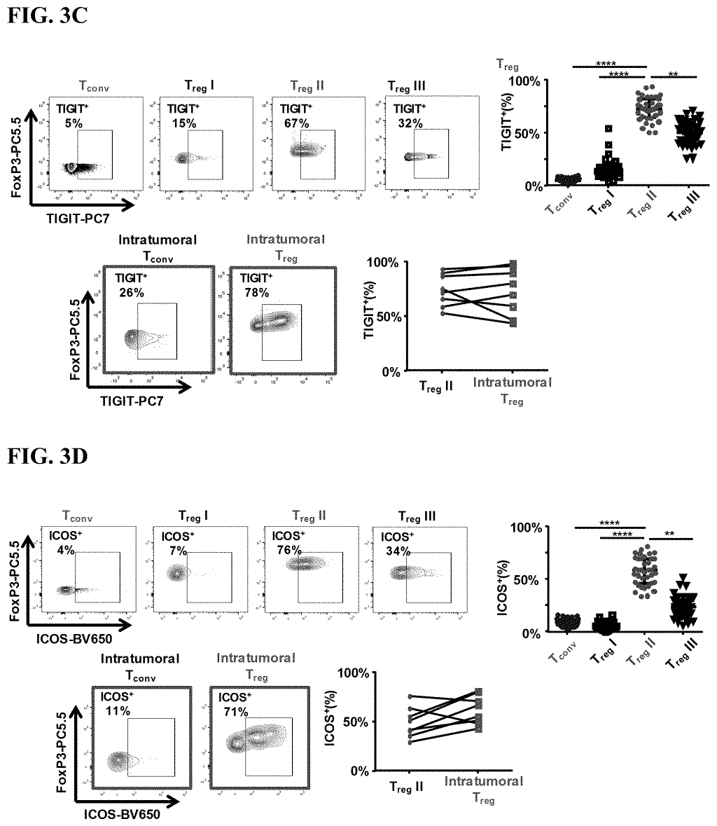

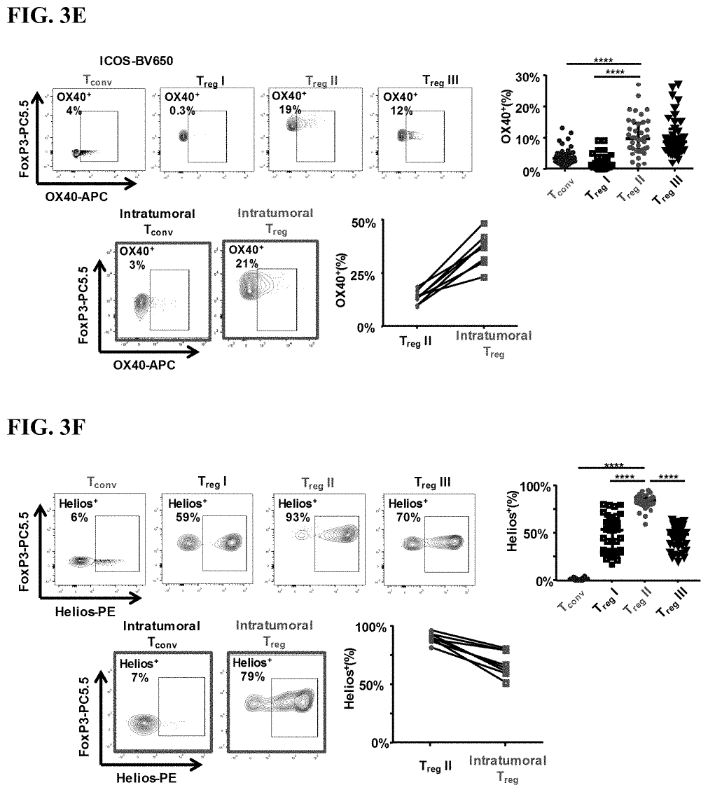

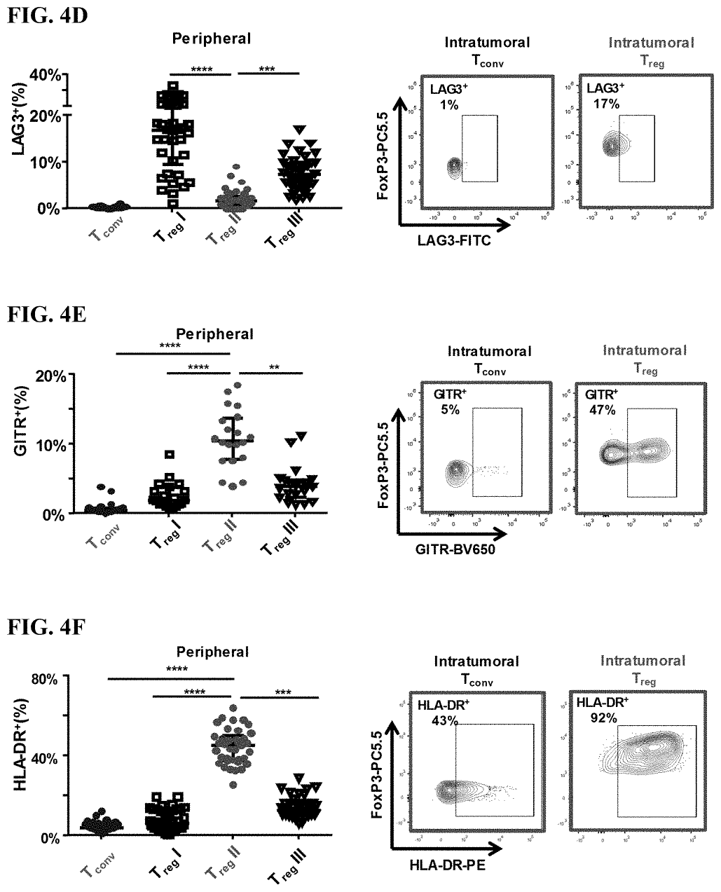

Next, the expression patterns of various important immune regulators in peripheral blood Treg cell subpopulations from newly diagnosed patients with BC (n=40) was examined. CD39 and CD73 are ATP metabolizing enzymes involved in the immunosuppressive functions of Treg cells (See for example Ref. 20). Data showed the percentage of CD39+ was significantly higher in Treg II than in Tconv, Treg I or Treg III cells (FIG. 3A), but the percentage of CD73+ was similarly low in Tconv cells and all Treg cell subpopulations (FIG. 4A). CD39 expression was similarly high between peripheral blood Treg II cells and intratumoral Treg cells from paired samples (FIG. 2A).

The expression of several immune co-inhibitory receptors, such as CTLA4, T cell immunoreceptor with Ig and ITIM domains (TIGIT), programmed cell death protein 1 (PD1), T cell immunoglobulin mucin (Tim)3 and lymphocyte activation gene (LAG)3 was examined in different Treg cell subpopulations. CTLA4 (FIG. 3B) and TIGIT (FIG. 3C) were higher in Treg II cells than in Tconv, Treg I or Treg III cells and were selectively expressed in intratumoral Treg cells (FIG. 3B-C). PD1 (FIG. 4B) and Tim3 (FIG. 4C) expressions were higher in Treg II than in Tconv cells but similar amongst all Treg cell subpopulations. In contrast, LAG3 expression was similarly low in Treg II, intratumoral Treg, and Tconv cells, but higher in Treg I and Treg III cells (FIG. 4D). In addition, we examined the expression of several immune co-stimulatory receptors, such as inducible T cell costimulator (ICOS), CD134 (OX40) and glucocorticoid-induced TNFR-related protein (GITR) in Treg subpopulations. ICOS (FIG. 3D) and OX40 (FIG. 3E) expression were higher in Treg II than in Tconv cells and were differentially expressed in intratumoral Treg cells (FIG. 3D-E). GITR was expressed higher in Treg II cells than in Tconv, Treg I or Treg III cells and was selectively expressed in intratumoral Treg cells (FIG. 4E).

Lastly, the expression of activation markers HLA-DR and Helios in Treg cell subpopulations was examined. The expression of HLA-DR (FIG. 4F) and Helios (FIG. 3F) in Treg II cells were higher than in Tconv, Treg I or Treg III cells and were differentially expressed in intratumoral Treg cells. Collectively, multiple immune-regulatory proteins are differentially expressed in peripheral blood Treg II cells and in paired intratumoral Treg cells from primary breast tumors, indicating that Treg II cells and intratumoral Treg cells have similar immune phenotype.

Peripheral Blood Treg II Cells Also have Similar Chemokine Receptor Expression Pattern with Intratumoral Treg Cells.

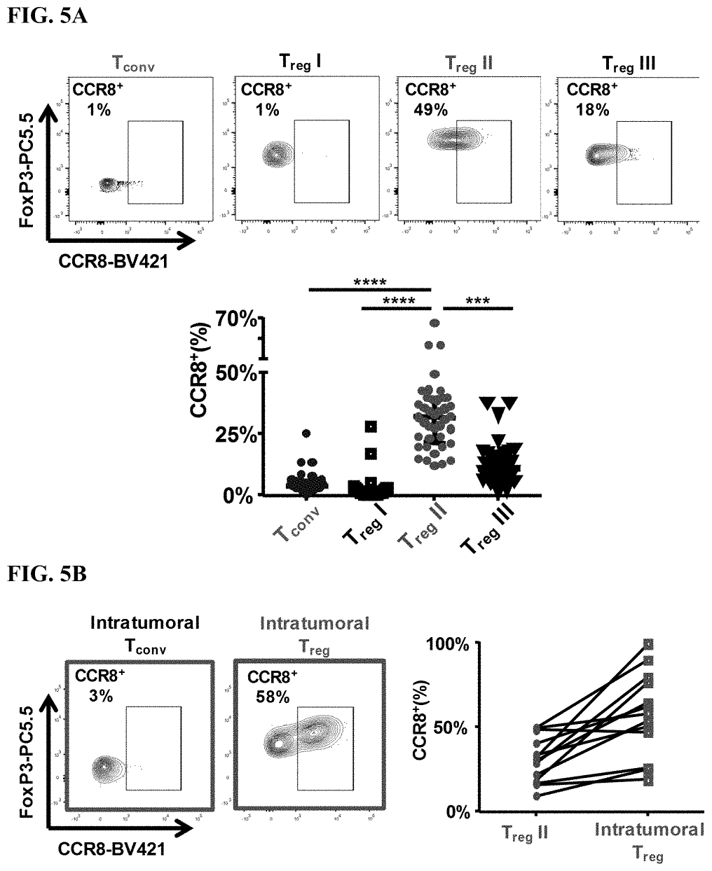

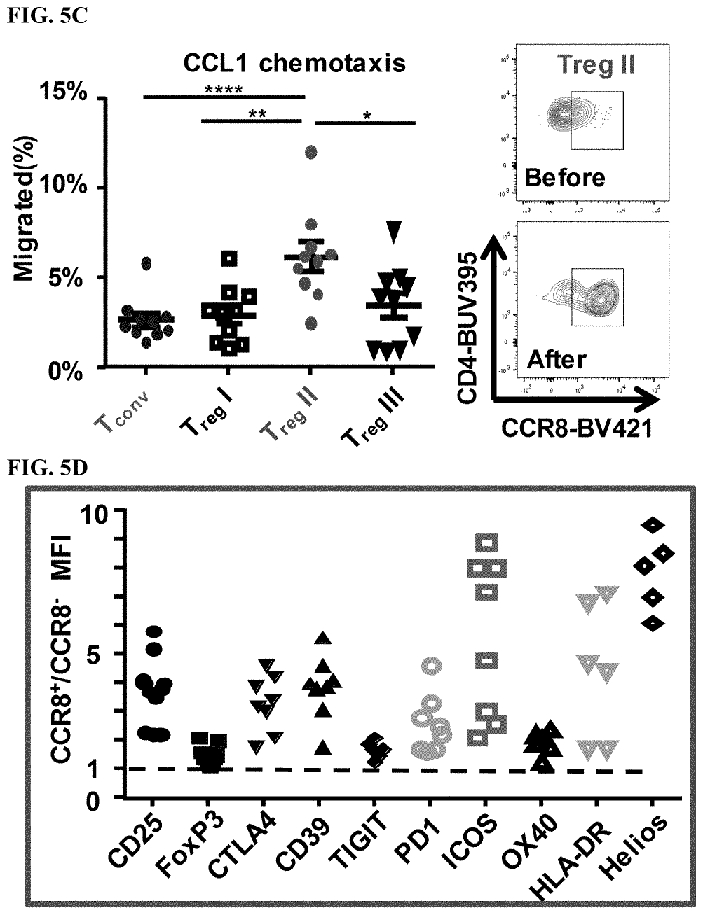

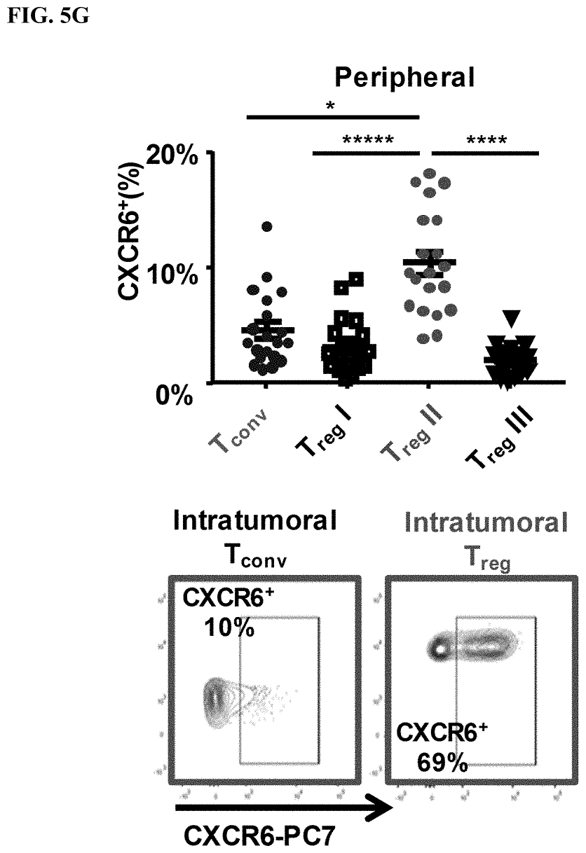

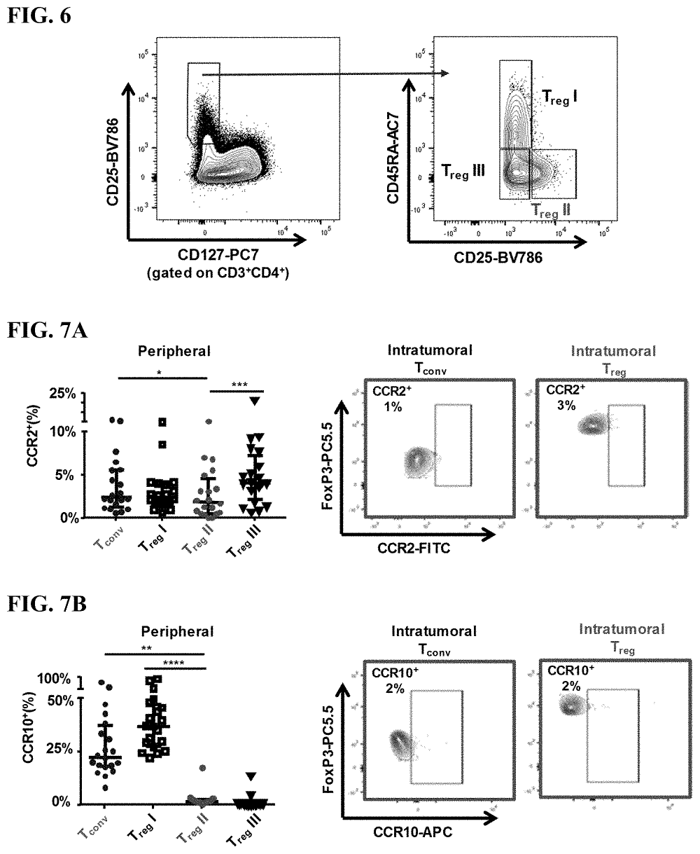

CCR8 has recently been identified as an important chemokine receptor on intratumoral Treg cells in several cancer types, including BC (See Refs. 21, 22). The expression of CCR8 and other chemokine receptors in peripheral blood Treg cell subpopulations was examined. The percentage of CC8⁺ was significantly higher in Treg II cells than in Tconv, Treg I or III cells (FIG. 5A). Consistent with these recent reports, CCR8 was selectively expressed in paired intratumoral Treg cells from primary breast tumors (FIG. 5B). To confirm the expression of CCR8 on peripheral Treg II cells has chemotactic potential, CCL1, the ligand of CCR8, induced chemotaxis of flow sorted Treg cell subpopulations (FIG. 6) from patients with BC was examined. CCL1-induced chemotaxis in Treg II cells was significantly higher than in Tconv, Treg I or III cells and CCR8⁺ enrichment after chemotaxis confirms that the migration was through the CCL1⁻CCR8 axis (FIG. 5C). Intratumoral CCR8⁺ Treg cells were shown to express higher CD25, FoxP3 and CTLA4 than CCR8− Treg cells (see for example Ref. 21). Data showed that intratumoral CCR8⁺ Treg cells also had higher expression of CD39, TIGIT, PD1, ICOS, OX40, HLA-DR and Helios than CCR8 Treg cells (FIG. 5D), supporting the notion that CCR8 may be not only a chemokine receptor but also a marker for activated Treg cells.

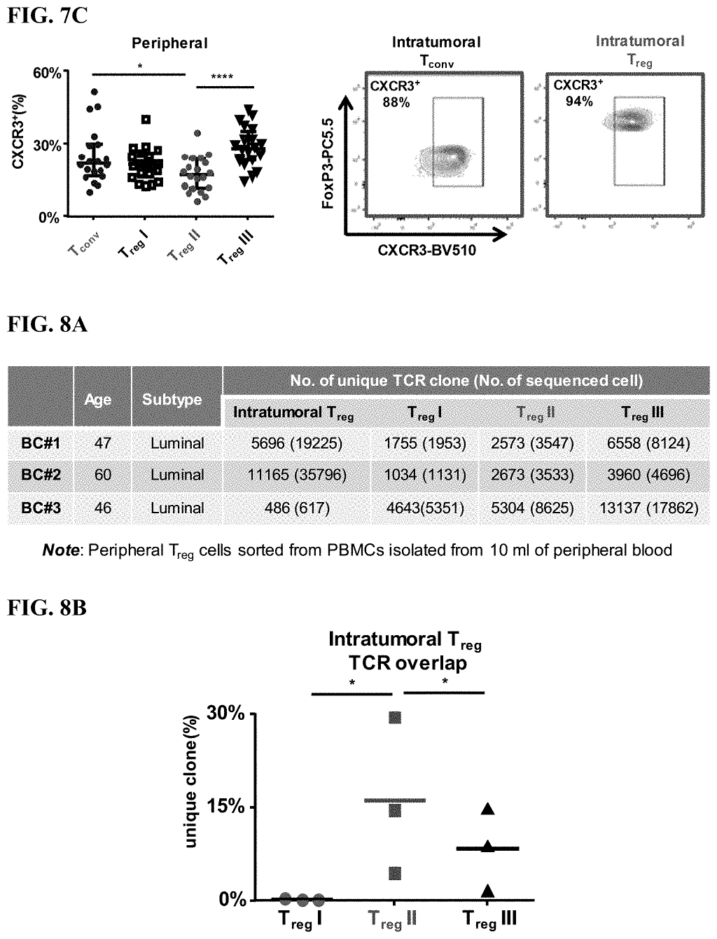

The expression of other chemokine receptors known to be involved in Treg trafficking, such as CCR2, CCR4, CCR5, CCR10, CXCR3 and CXCR6 23 was examined in peripheral Treg cell subpopulations from patients with BC. Amongst these receptors, CCR4 (FIG. 5E), CCR5 (FIG. 5F) and CXCR6 (FIG. 5G) were significantly higher in Treg T cells than in Tconv, Treg I or II cells and were also differentially expressed in intratumoral Treg cells. In contrast, the percentages of CCR2⁺, CCR10⁺ and CXCR3⁺ were significantly lower in Treg II than in Tconv cells and these chemokine receptors were not selectively expressed in intratumoral Treg cells (FIG. 7A-C). These findings reveal that peripheral Treg II cells have similar chemokine receptor expression pattern with intratumoral Treg cells.

Intratumoral Treg Cells Share More TCR Clonal Overlap with Peripheral Treg II Cells.

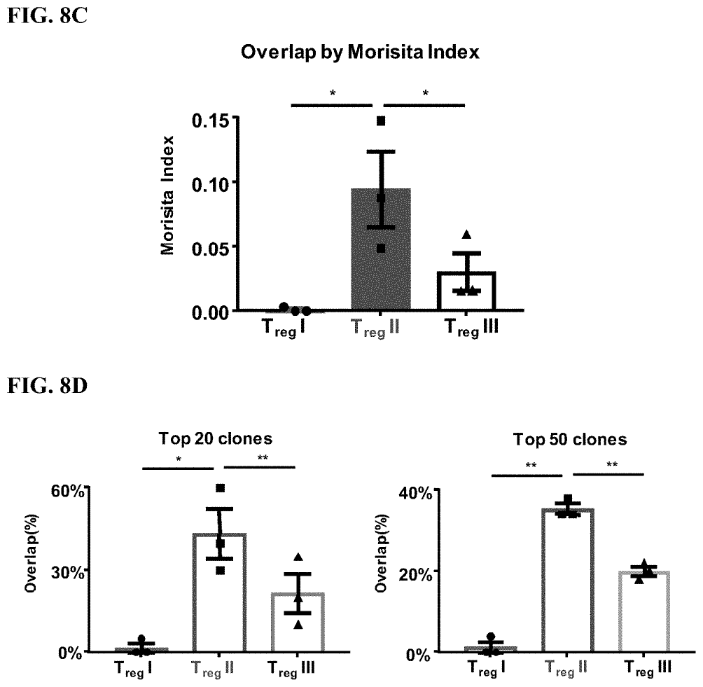

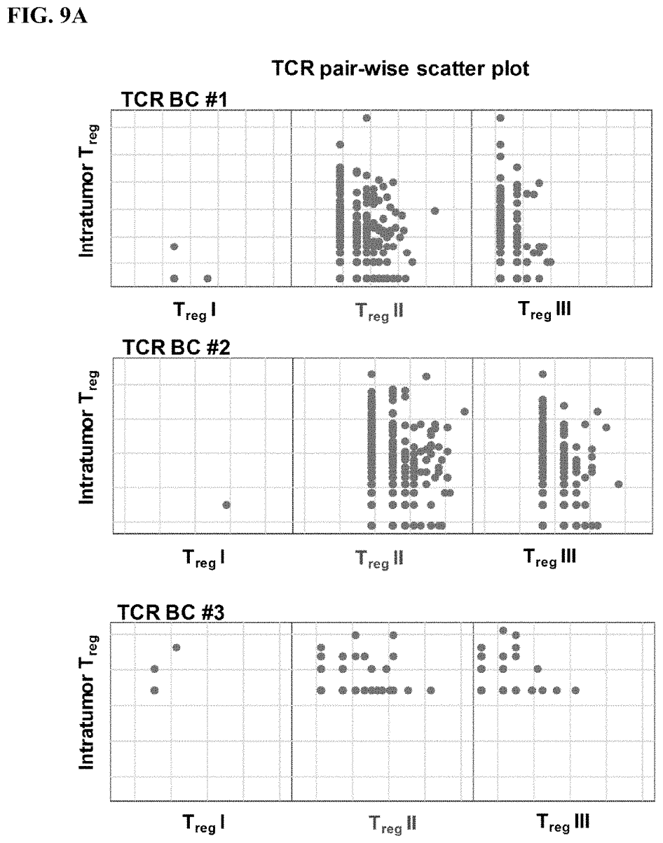

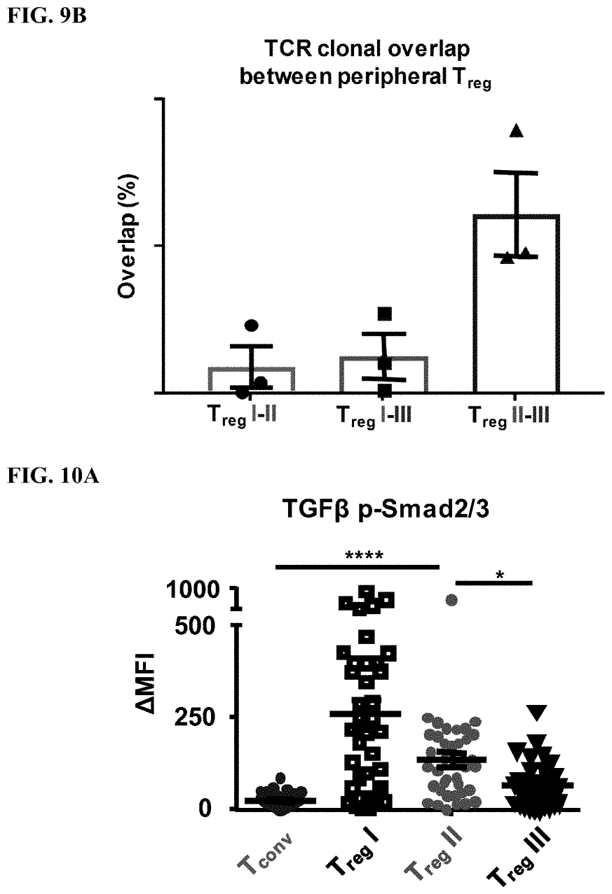

To further support that peripheral blood Treg II cells have higher tumor infiltration tendency than Treg I or III cells, the T cell receptor (TCR) repertoires were compared between sorted peripheral Treg cell subpopulations and paired intratumoral Treg cells (CD4⁺CD25⁺CD127⁻) from three patients with BC (identified unique TCR clone numbers listed in FIG. 8A). Remarkably, the percentage of intratumoral Treg TCR clones which overlapped with Treg II cells was significantly higher than with Treg II cells, and the overlap was very low with Treg I cells (FIG. 8B-C) (TCR pair-wise scatter plots showing the overlapping clones in FIG. 9A). In addition, about 40% of top 20 or top 50 intratumoral Treg TCR clones overlapped with peripheral Treg II cells (FIG. 8D). Notably, the data demonstrated very low TCR overlap between Treg I and Treg II or II cells (FIG. 9B), suggesting that Treg I cells may not convert to Treg II or III cells. These findings suggest that peripheral Treg II and III cells may both infiltrate breast tumors, whereas Treg I cells may not tend to infiltrate tumors at all.

Signaling Responsiveness to Individual Cytokines in Peripheral Blood Treg II Cells Correlates with Clinical Outcome of Patients with BC.

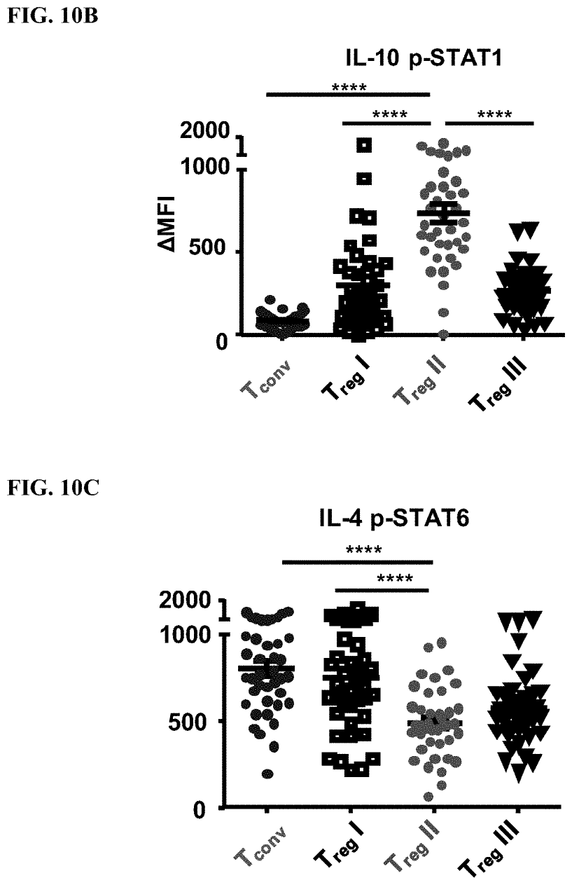

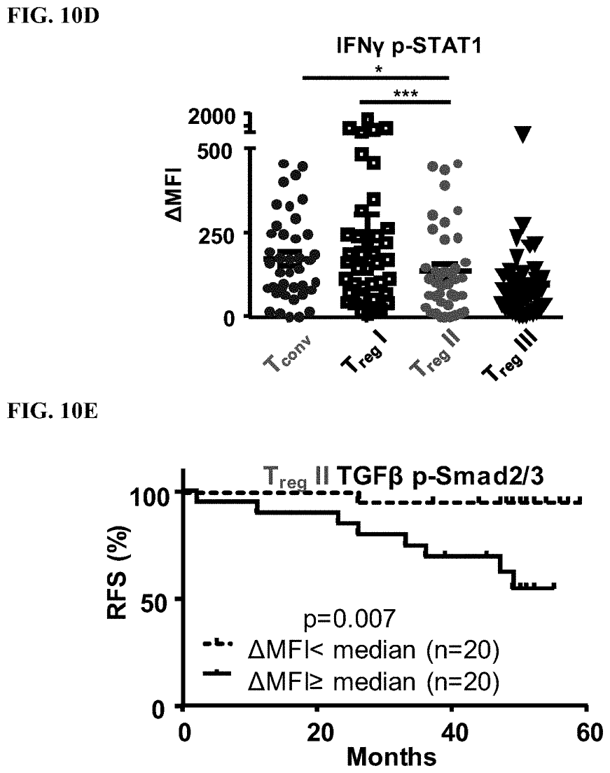

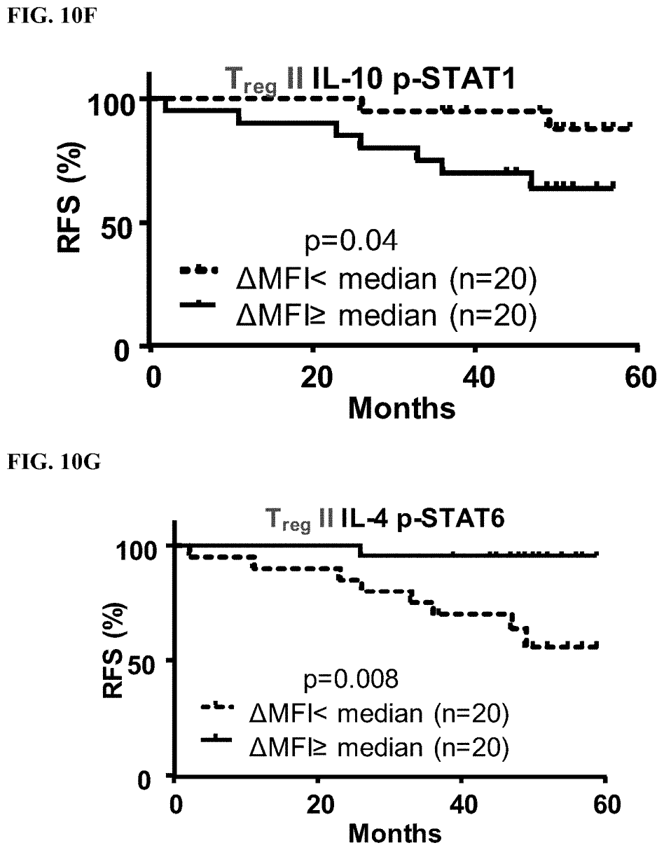

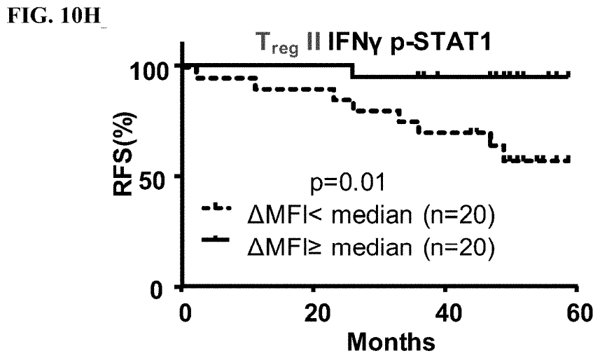

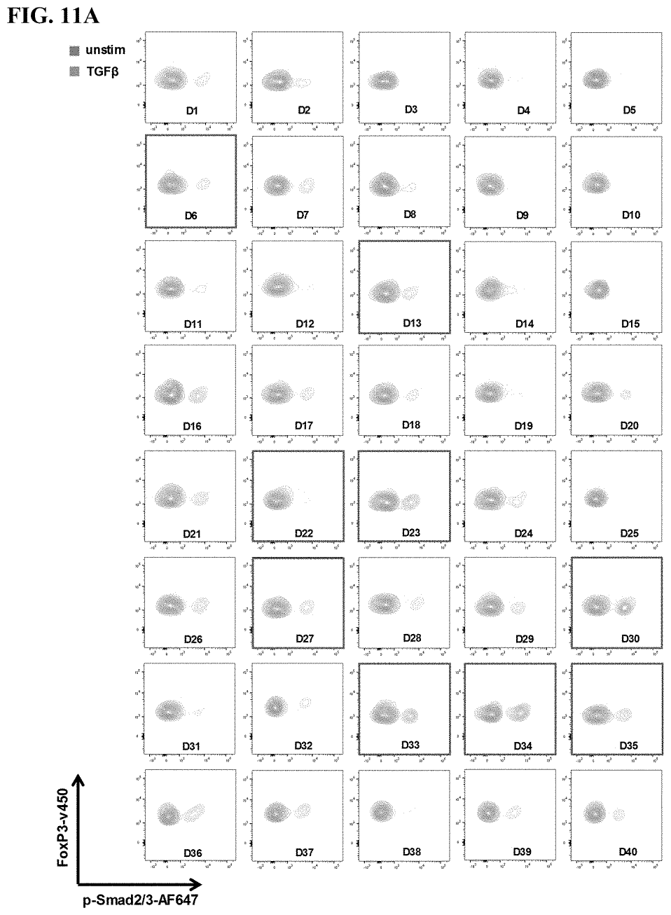

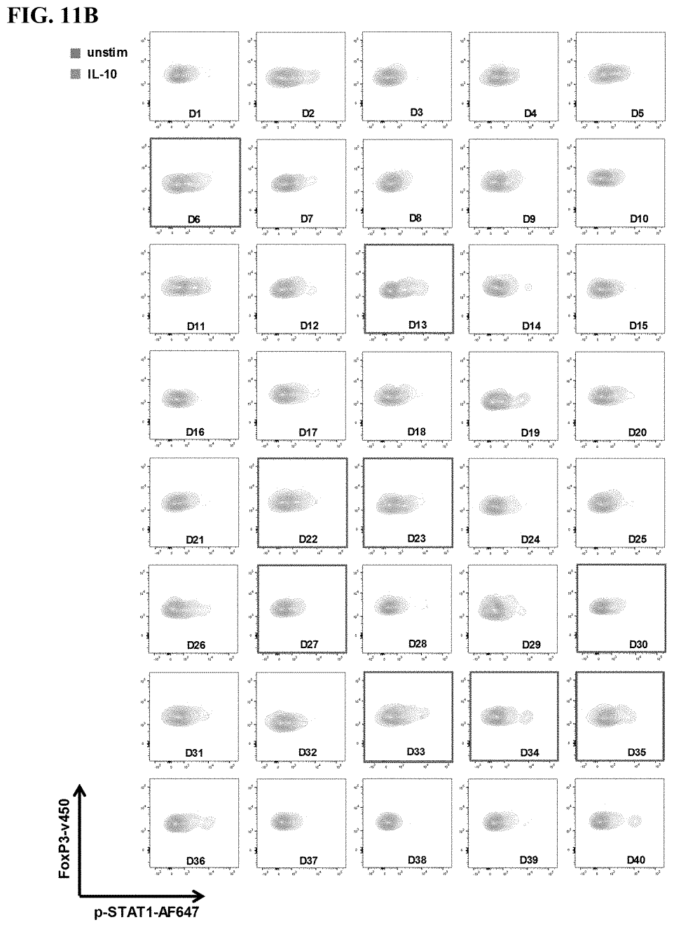

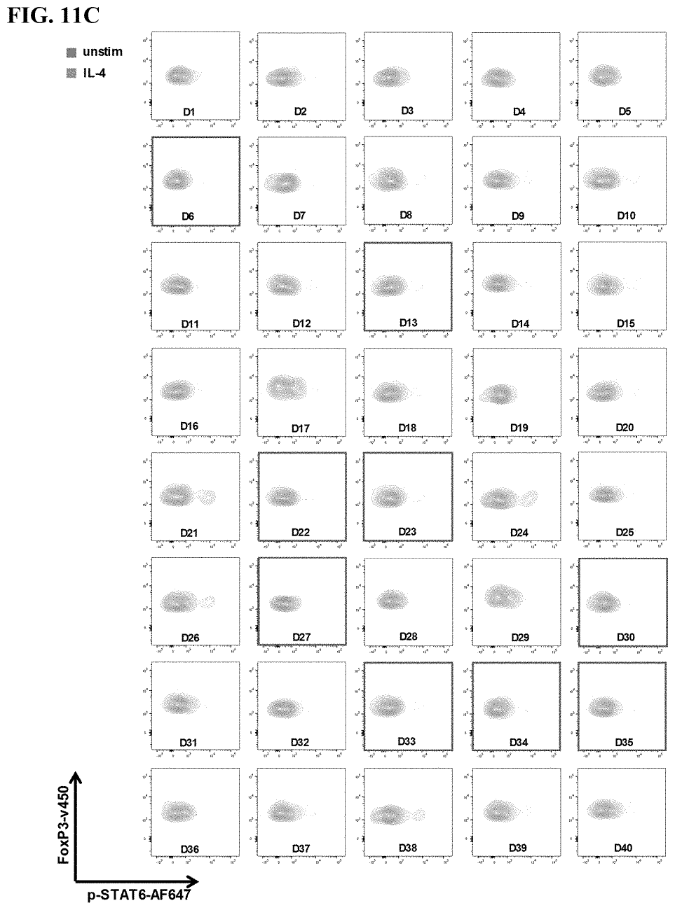

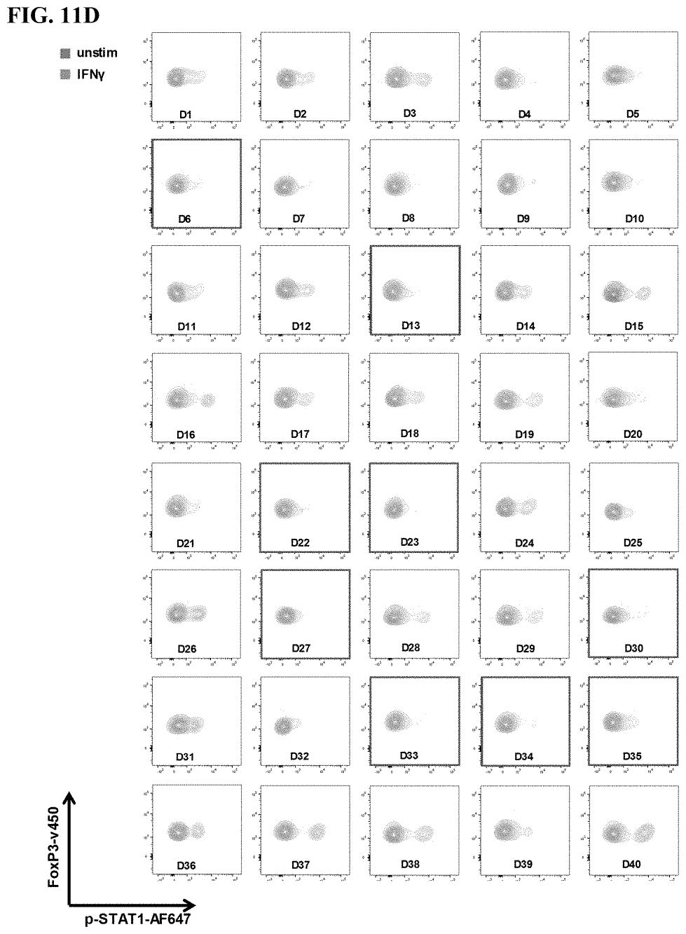

While the immunosuppressive mechanisms of Treg cells include production of immunosuppressive cytokines, the ability of Treg cells to respond to cytokines in cancer remains largely unexplored. The clinical significance of cytokine signaling induced by immunosuppressive cytokine TGF-β or IL-10, Th2 cytokine IL-4, and Th1 cytokine IFN-γ in peripheral blood Treg cell subpopulations from patients with BC was investigated. Only patients with blood collected at diagnosis before surgery or any therapy and had been clinically followed for at least 36 months were selected (clinical and pathological characteristics are summarized in Table 1). Median follow-up time of patients with BC (n=40) was 49 months (range, 36-59 months). The data demonstrated that TGFβ-induced phosphorylation of Smad2/3 (p-Smad2/3) (FIG. 11A) was significantly higher in Treg II than in Tconv or Treg III cells (FIG. 10A). Additionally, IL-10-induced p-STAT1 (FIG. 11B) were significantly higher in Treg II than in Tconv, Treg I or Treg III cells (FIG. 10B). In contrast, Th2 cytokine IL-4-induced p-STAT6 (FIG. 11C) and Th1 cytokine IFNγ-induced p-STAT1 (FIG. 11D) were significantly lower in Treg II than in Tconv or Treg I cells (FIG. 10C-D). Thus, the results herein demonstrate that Treg II cells may be more sensitive to immunosuppressive cytokines and less sensitive to Th1/2 cytokines as compared to Tconv cells or the other Treg cell subpopulations.

TABLE 1
Patient Characteristics

| | Discovery Cohort | | Validation Cohort | |
|---|---|---|---|---|
| Characteristics | N = 40 (%) | | N = 38 (%) | |
| Age-yr | | | | |
| Median | 52 | | 53 | |
| Range | 28-79 | | 27-77 | |
| Tumor stage- no. (%) | | | | |
| DCIS | 0 | (0) | 2 | (5) |
| T1 | 18 | (45) | 16 | (42) |
| T2 | 16 | (40) | 17 | (45) |
| T3 | 4 | (10) | 3 | (8) |
| Unknown | 2 | (5) | 0 | (0) |
| Grade- no. (%) | | | | |
| G1 | 4 | (10) | 5 | (13) |
| G2 | 24 | (60) | 22 | (58) |
| G3 | 12 | (30) | 11 | (29) |
| Nodal status- no. (%) | | | | |
| N0 | 22 | (55) | 28 | (74) |
| N1-3 | 15 | (37.5) | 10 | (26) |
| Unknown | 3 | (7.5) | 0 | (0) |
| Subtype- no. (%) | | | | |
| Luminal | 33 | (82.5) | 32 | (84) |
| HER2 | 4 | (10) | 4 | (10.5) |
| Triple negative | 3 | (7.5) | 2 | (5.5) |

Experiments were conducted to investigate if cytokine signaling responses in peripheral blood Treg cells correlate with clinical outcome. Kaplan-Meier survival analysis and log-rank test were used to determine the relationship between cytokine signaling responsiveness in peripheral Treg subpopulations and relapse-free survival (RFS). Patients with BC were divided into two populations using the median cytokine signaling response (ΔMFI) as the cut-off value. Indeed, the data showed found that patients with BC with above median cytokine signaling response to immunosuppressive cytokine TGFβ (p=0.007) (FIG. 10E) or IL-10 (p=0.04) (FIG. 10F) in Treg II cells had worse RFS. In contrast, patients with BC with below median cytokine signaling response to Th2 cytokine IL-4 (p=0.008) (FIG. 10G) or Th1 cytokine IFNγ (p=0.01) (FIG. 10H) in Treg II cells had worse RFS. Notably, no significant correlation between cytokine signaling in Treg I, Treg III or Tconv cells and clinical outcome was observed (data not shown). Taken together, these findings demonstrate that cytokine signaling responsiveness in peripheral blood Treg II cells reflects the immune status of patients with BC at diagnosis: patients with higher signaling response to immunosuppressive cytokines but lower signaling response to Th1/2 cytokines have higher risk of future relapse.

Combined Cytokine Signaling Index in Peripheral Blood Treg II Cells Reflects their Immunosuppressive Potential and Strongly Correlates with Clinical Outcome.

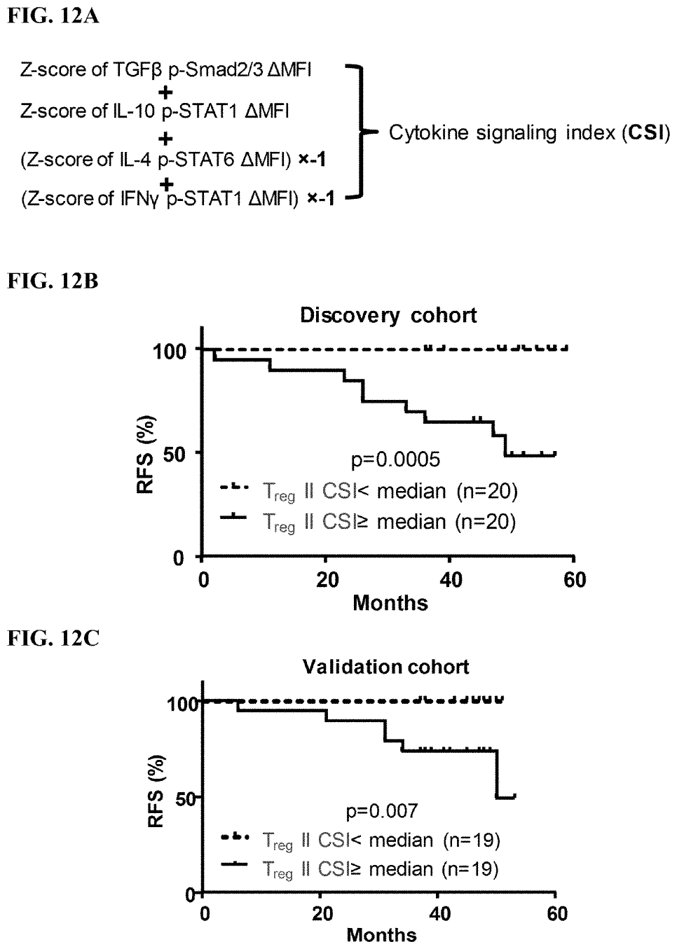

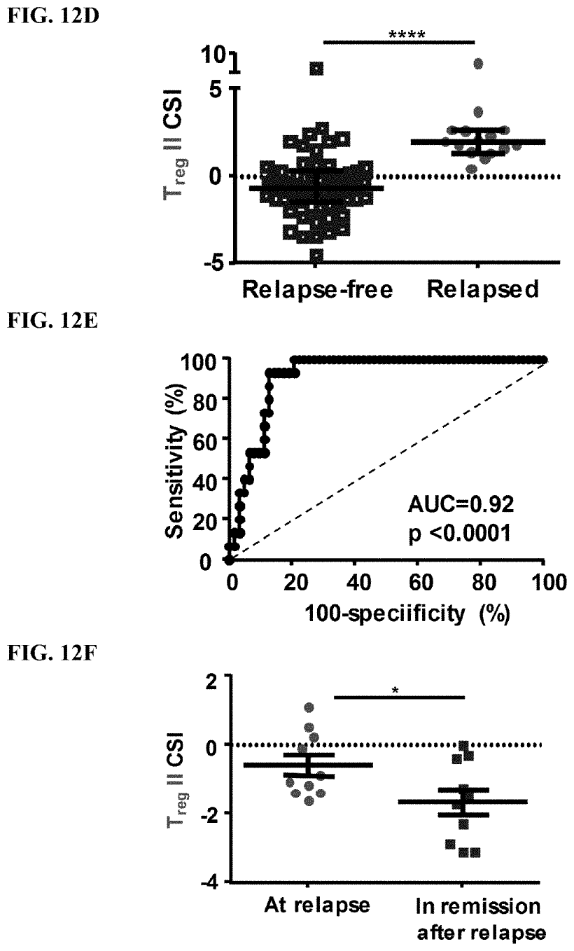

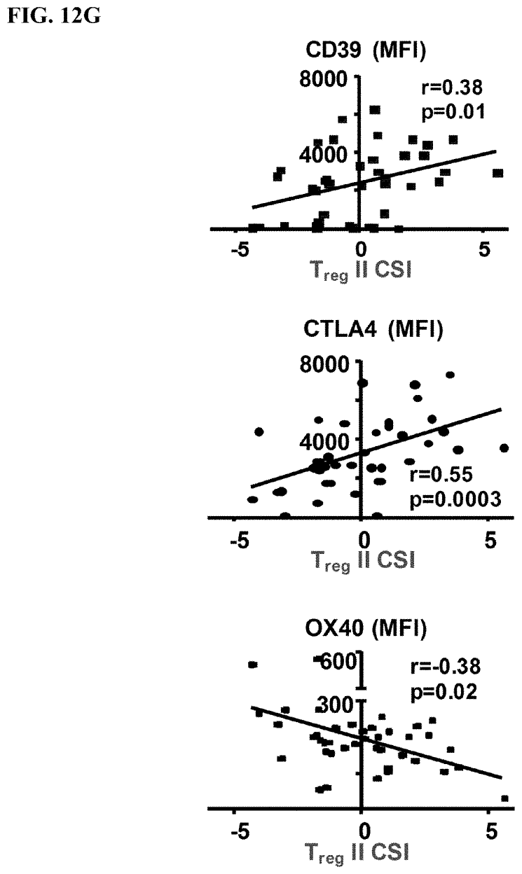

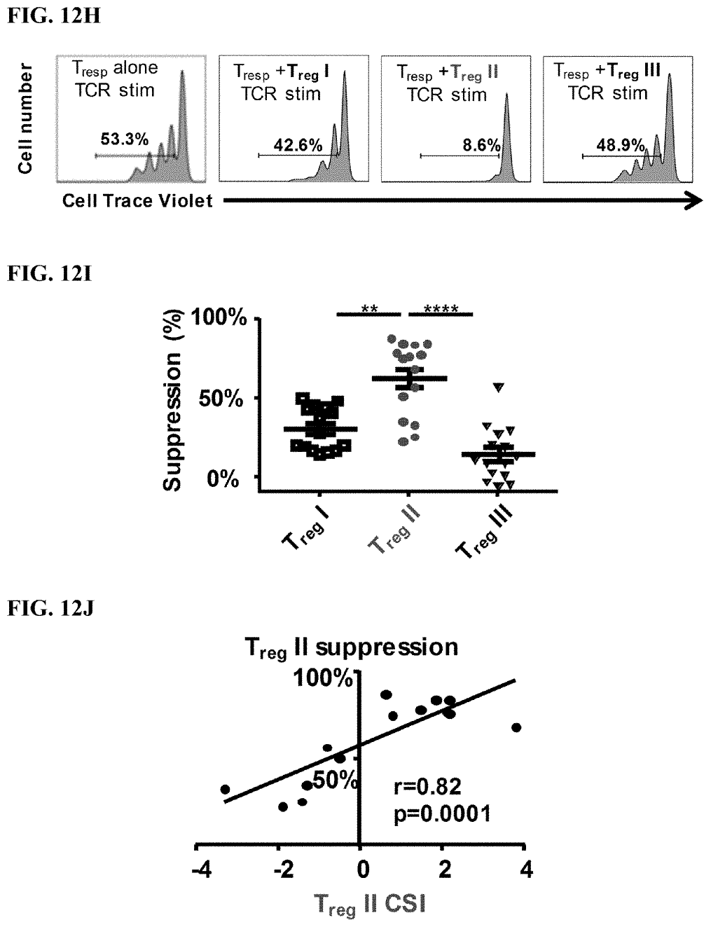

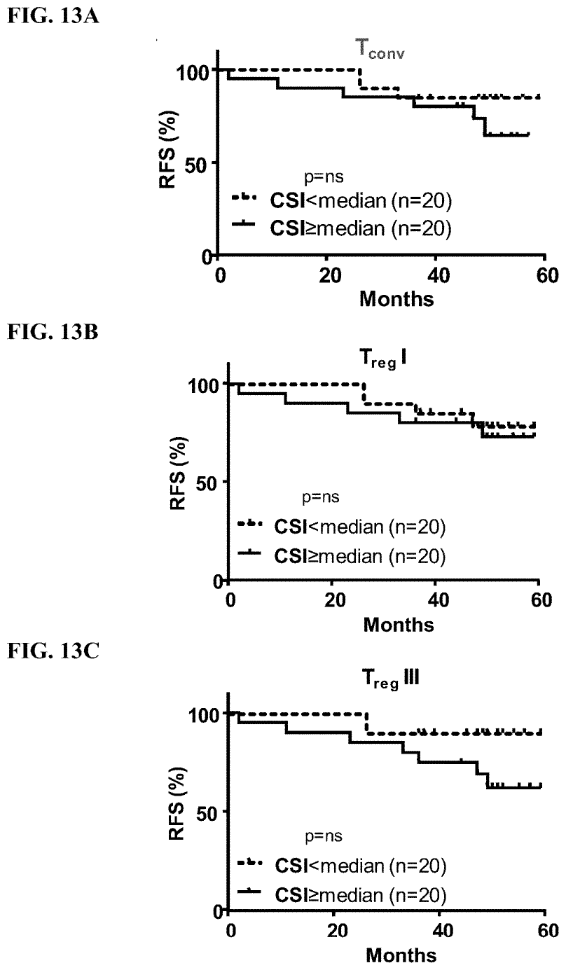

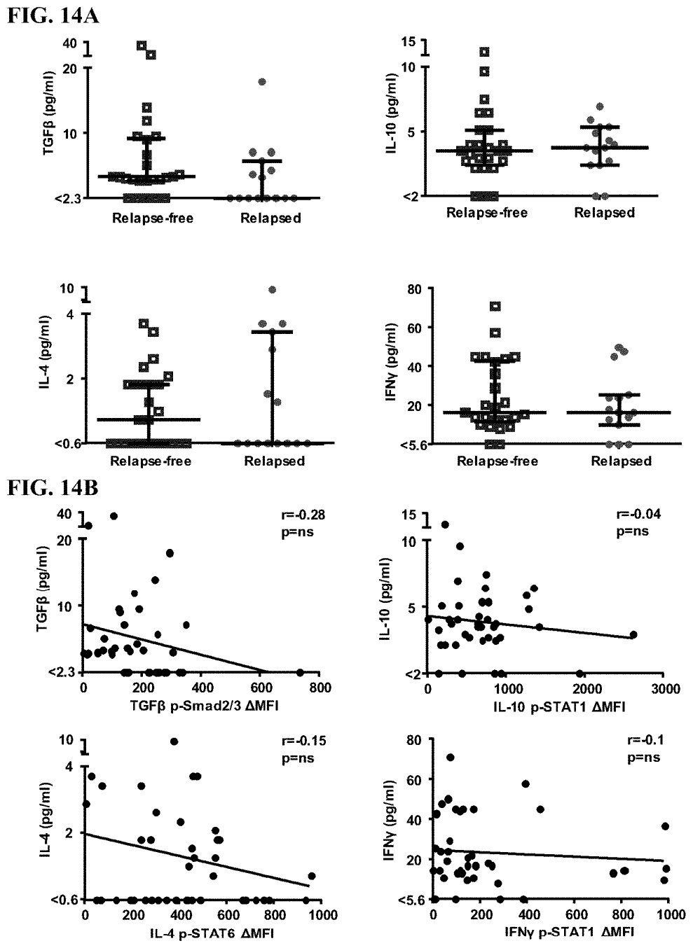

Since cytokine signaling responses to immunosuppressive vs. immunostimulatory cytokines have opposing clinical outcome correlations, a cytokine signaling index (CSI) was created to better represent the balance of signaling responses to these opposing cytokines in Treg cells using the sum of Z-score of TGFβ-induced p-Smad2/3, IL-10-induced p-STAT1 and Z-score of IL-4-induced p-STAT6 and IFNγ-induced p-STAT1 multiplied by −1 (FIG. 12A). Patients with BC (n=40) were divided into two populations using median Treg II CSI as the cut-off value and Kaplan-Meier analysis was performed to determine the relationship between Treg II CSI and RFS. The data showed that BC patient with above median Treg II CSI had significantly worse RFS (p=0.0005) while none of the patients with below median Treg II CSI experienced relapse (FIG. 12B). Notably, there was no correlation between CSI of Treg I, III or Tconv cells and RFS (FIG. 13A-C).

To evaluate the robustness of Treg II CSI in predicting the risk of future relapse of patients with BC, the clinical significance of cytokine signaling responses in peripheral Treg II cells was confirmed using an independent validation cohort of patients with BC (n=38). Again, only patients with blood collected at diagnosis before surgery or any therapy and had been clinically followed for at least 36 months were selected (clinical and pathological characteristics are summarized in Table 1). The median follow-up time was 45 months (range, 37-53 months). Patients with BC in the validation cohort who have above median Treg II CSI had worse RFS (p=0.007) and none of the patients with below median Treg II CSI experienced relapse (FIG. 12C). We combined patients with BC from the discovery and validation cohorts, re-calculated the Treg II CSI using Z-score of the combined cohort, and found that Treg II CSI of relapsed patients was significantly higher than relapse-free patients (FIG. 12D). Importantly, Treg II CSI was independent of patients' age, tumor stage, tumor grade, nodal status or subtype (Table 2). The prognostic potential of using cytokine signaling response in peripheral Treg II cells to predict future relapse was also evaluated by receiver operating characteristic (ROC) analysis. Treg II CSI achieved an area under the curve (AUC) of 0.92 (95% CI 0.86-0.97, p<0.0001), with 93% sensitivity and 87% specificity to predict future relapse when Treg II CSI at 0.88 was used as the cut-off (FIG. 12E).

TABLE 2
Correlation of clinicopathological characteristics
with Treg II cytokine signaling index (CSI)

| | | Cytokine signaling index (CSI) | | | | |
|---|---|---|---|---|---|---|
| | | Below | | Above | | p-value |
| Clinical factors | | median | | median | | (Chi-square) |
| Age, yr | | | | | | |
| | ≤53 | 25 | (32%) | 17 | (21.8%) | 0.07 |
| | >53 | 14 | (18%) | 22 | (28.2%) | (n = 78) |
| Tumor stage | | | | | | |
| | DCIS | 0 | (0%) | 2 | (2.6%) | 0.23 |
| | T1 | 20 | (26.3%) | 14 | (18.4%) | (n = 76) |
| | T2 | 17 | (22.4%) | 16 | (21.1%) | |
| | T3 | 2 | (2.6%) | 5 | (6.6%) | |
| Grade | | | | | | |
| | G1 | 4 | (5.1%) | 5 | (6.4%) | 0.49 |
| | G2 | 21 | (27%) | 25 | (32%) | (n = 78) |
| | G3 | 14 | (18%) | 9 | (11.5%) | |
| Nodal status | | | | | | |
| | N0 | 26 | (34.7%) | 24 | (32%) | 0.74 |
| | N1-3 | 12 | (16%) | 13 | (17.3) | (n = 75) |
| Subtype | | | | | | |
| | Luminal | 32 | (41%) | 33 | (42.3%) | 0.9 |
| | HER2 | 4 | (5.1%) | 4 | (5.1%) | (n = 78) |
| | Triple negative | 3 | (3.9%) | 2 | (2.6%) | |

Next, experiments were conducted to investigate whether plasma levels of these cytokines in patients with BC correlate with clinical outcome and/or reflect peripheral Treg II cell cytokine signaling response. The data showed that plasma levels of TGFβ, IL-10, IL-4 and IFNγ were similar between relapse-free and relapsed patients with BC (FIG. 14A), and no significant association between plasma levels of these cytokines with their signaling responses in peripheral Treg II cells (FIG. 14B).

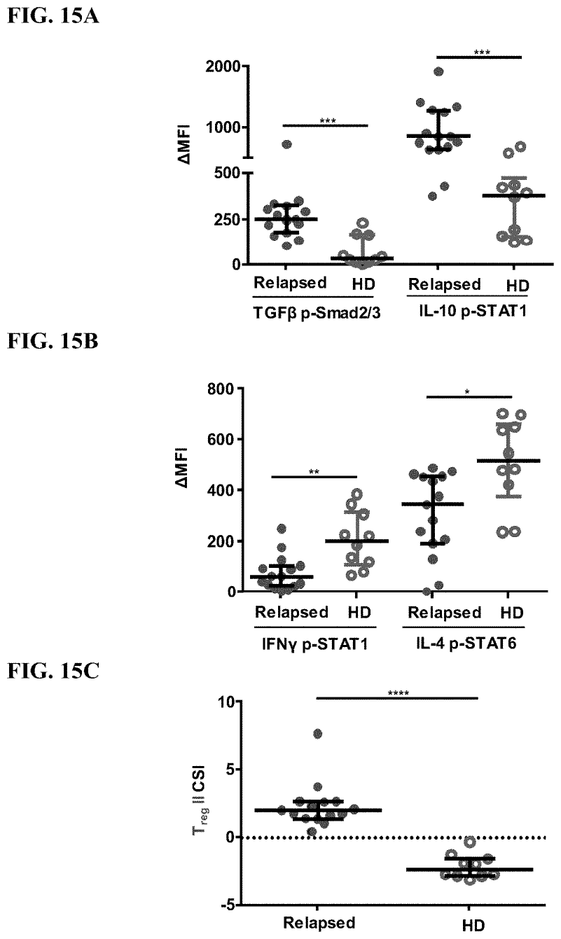

To understand how cytokine signaling responsiveness may evolve overtime inpatients from diagnosis to relapse, signaling responsiveness to TGFβ, IL-10, IFNγ and IL-4 in peripheral Treg II cells were compared between relapsed patients with BC and age-matched healthy donors (n=10). In healthy donors, signaling responses in Treg II cells to TGFβ and IL-10 were lower (FIG. 15A), but signaling responses to IFNγ and IL-4 were higher (FIG. 15B) as compared to relapsed patients with BC, leading to a lower Treg II CSI in healthy donors than in relapsed patients with BC (FIG. 15C). Furthermore, the Treg II CSI was determined and compared between patients with BC with blood collected at relapse (n=10) and patients who achieved and remained in remission for at least 36 months after their most recent relapse (n=10). Treg II CSI was significantly higher in patients with BC at relapse than in patients who are in remission (FIG. 12F). Taken together, these data suggest that Treg II CSI changes over time, reflecting the underlying disease state in patients with BC.

Experiments were conducted to examiner whether Treg II CSI correlates with expression levels of other immunoregulatory proteins on Treg II cells. CSI positively correlated with levels of CD39 and CTLA4 (FIG. 12G) in Treg II cells. In contrast, a negative correlation was found between CSI and OX40 level in Treg II cells (FIG. 12G), consistent with the reported negative regulation of OX40 on Treg cell activity (See for example Ref. 24).

To establish CSI as an indicator of the immunosuppression potential of Treg cells, the in vitro suppressive activity of peripheral blood Treg cell subpopulations from patients with BC was examined. Autologous peripheral blood Treg cell subpopulations and responder T cells (CD4+CD45RA+CD25−, Tresp) from patients with BC were sorted and cocultured. Treg II cells potently suppressed TCR stimulation-induced proliferation of Tresp cells (FIG. 12H) and the suppressive activity of Treg II cells was higher than Treg I or Treg III cells (FIG. 12I). The data demonstrated a significant positive correlation between CSI and the suppressive activity of Treg T cells (FIG. 12J), suggesting CSI indeed reflects the suppressive potential of peripheral blood Tregs from patients with BC.

Cytokine Signaling Index (CSI) of Treg II Cells Reflects the Immunosuppressive Potential of Intratumoral Treg Cells.

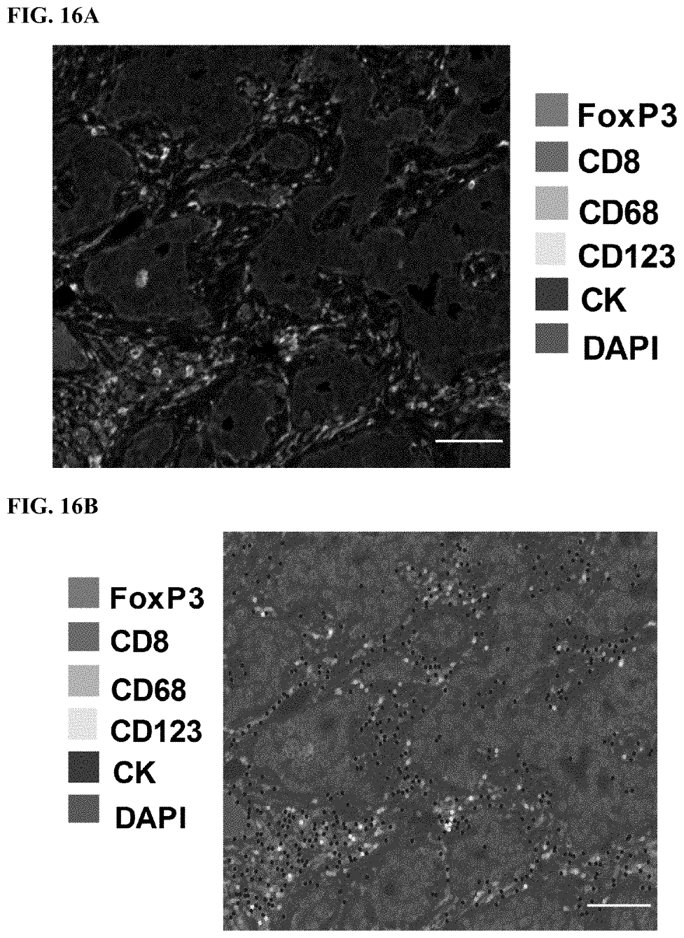

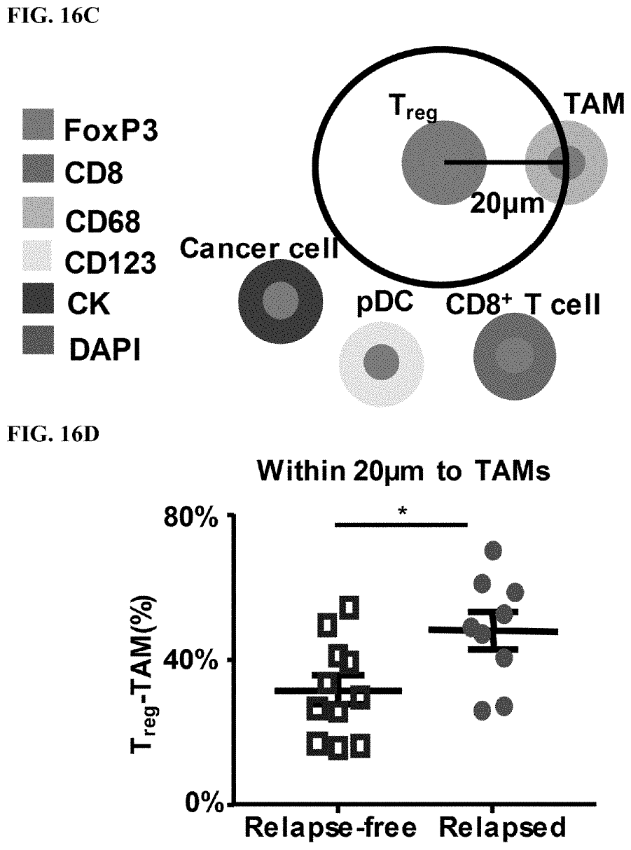

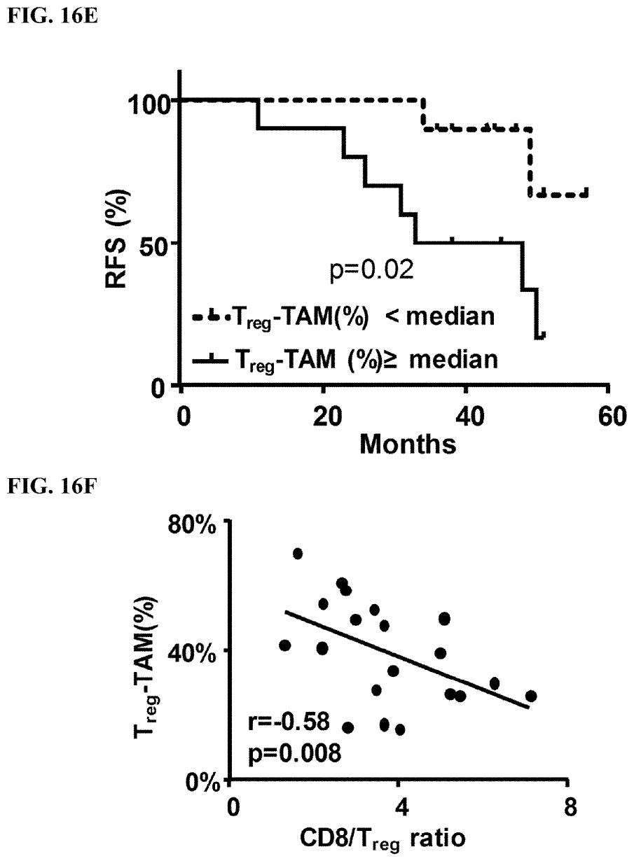

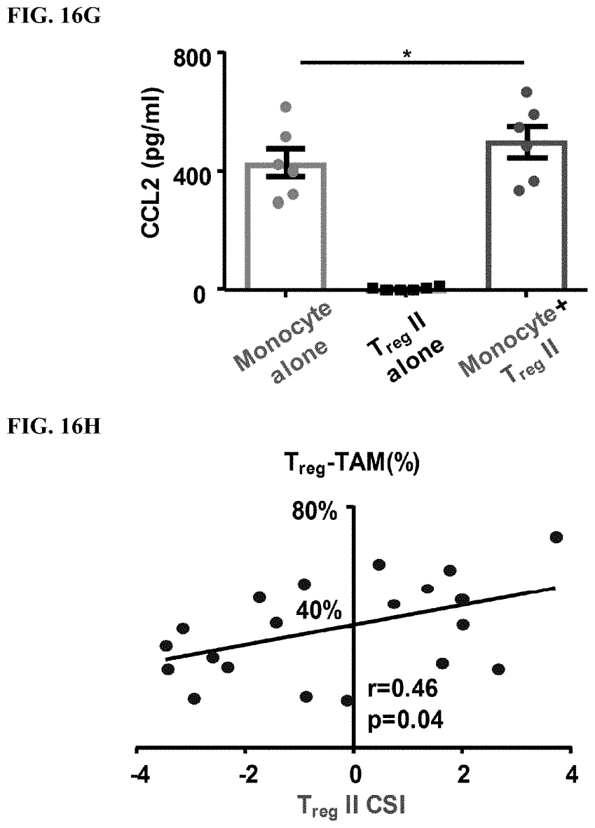

Since Treg cells exert their suppressive activity via various contact-dependent mechanisms, experiments were conducted to investigate whether higher CSI of peripheral Treg T cells indicates intratumoral immunosuppressive activity. Multiplex immunofluorescence staining was performed to identify Treg cells (FoxP3), CD8+ T cells (CD8), tumor-associated macrophages (TAMs) (CD68), plasmacytoid dendritic cells (pDCs) (CD123) and cancer cells (CK), within the TME of untreated primary breast tumors (n=20) (FIG. 16A). Slides were scanned and images were quantitatively examined for interactions between Treg and other cells using cell spatial coordinates obtained from the corresponding phenotype maps (FIG. 16B). It was hypothesized that the interplay between Tregs with other cells creates an immunosuppressive TME. Utilizing an accepted assumption that two cells are more likely to be interacting if the distance between their nuclei is less than 20 μm 25, the percentages of Treg cells that were less than 20 μm from CD8+ T cells, TAMs, pDCs or cancer cells (FIG. 16C) was quantified. Results showed that the percentage of Treg cells less than 20 μm from TAMs (% Treg-TAM) was significantly higher in the primary tumors from patients who later relapsed than from patients who remained relapse-free (FIG. 16D). Kaplan-Meier survival analysis and log-rank test confirmed that patients with BC with above median % Treg-TAM in their tumors had worse RFS (FIG. 16E).

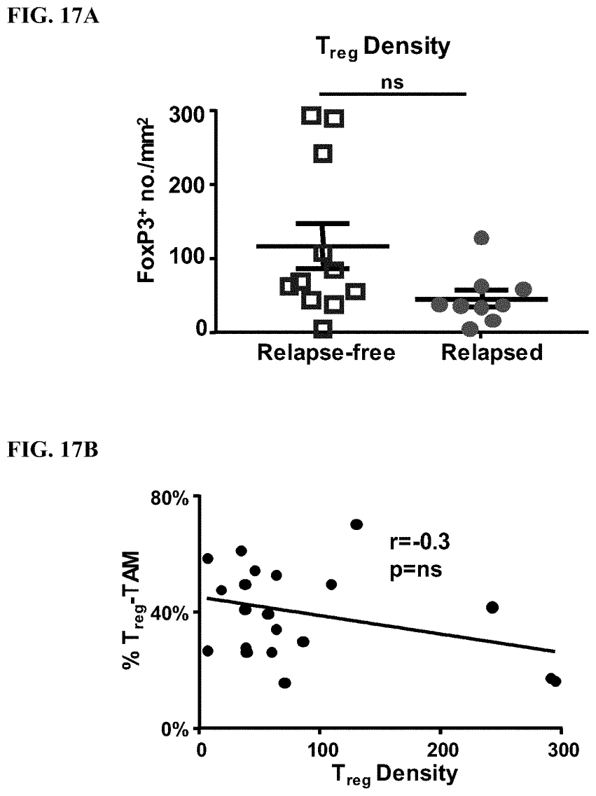

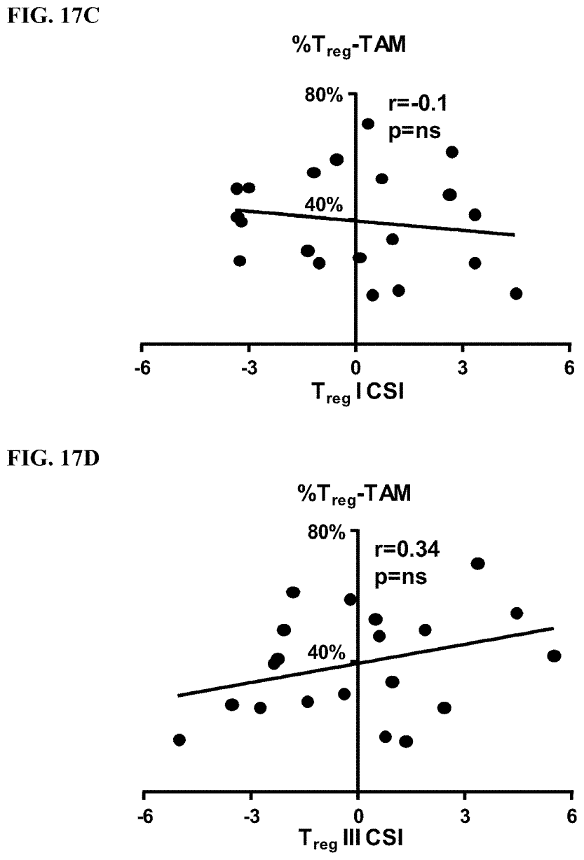

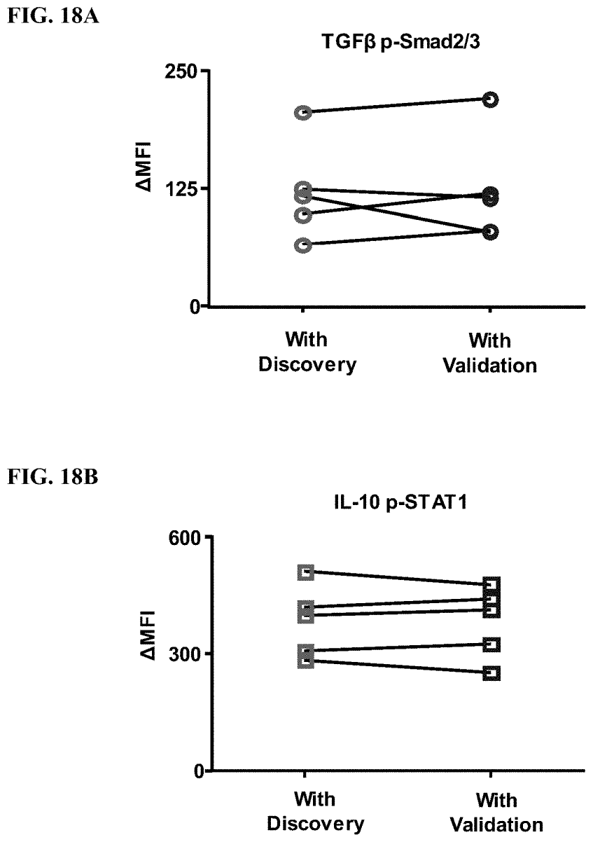

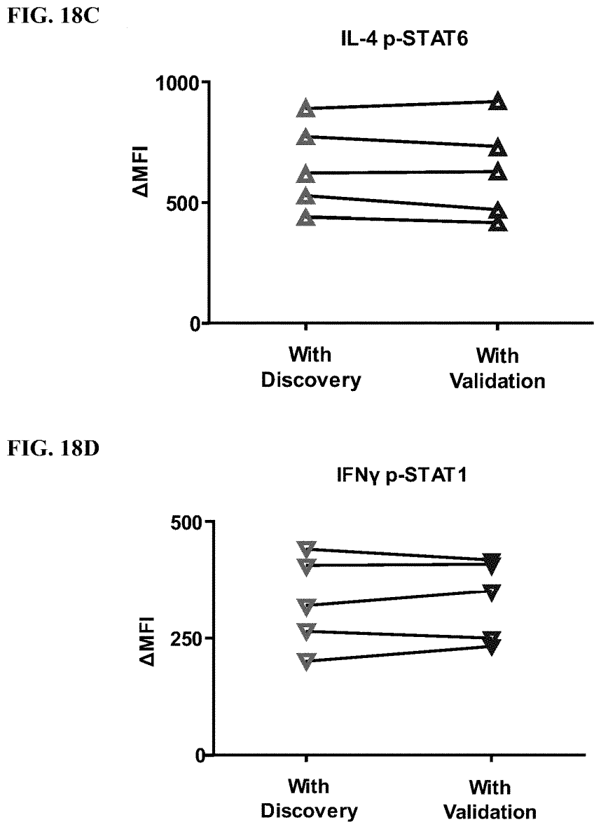

CD8/Treg ratio within tumors is accepted as a robust and reliable measure of its immune status: low CD8/Treg ratio is associated with an immunosuppressive TME and poor clinical outcome (See for example Refs. 9, 26, 27). The data herein showed a clear negative correlation between % Treg-TAM with CD8/Treg ratio (FIG. 16F). These data suggest that potential interactions between Treg cells and TAMs create a more immunosuppressive TME, leading to poor clinical outcome. Importantly, Treg cell density within primary tumors was similar between relapsed and relapse-free patients (FIG. 17A) and independent of % Treg-TAM (FIG. 17B). To further investigate the effects of Treg-TAM interactions, peripheral blood monocytes (CD14+) from patients with BC were cocultured with autologous peripheral Treg II cells and it was found that monocytes/macrophages production of CCL2, an important tumor infiltration chemokine for monocytes, was increased by Treg II cells, while Treg II cells alone did not produce CCL2 (FIG. 16G). Lastly, CSI of peripheral blood Treg II cells (FIG. 16H), but not of Treg I (FIG. 17C) or III cells (FIG. 17D), was positively associated with intratumoral % Treg-TAM. These data demonstrate that higher peripheral blood Treg II CSI may reflect more frequent interaction between Treg cells and TAMs within tumors, and intratumoral Treg-TAM interactions may be important for the development of an immunosuppressive TME.

Discussion

Chemokine receptors on Treg cells play a pivotal role in the recruitment of Treg cells into tumors: CCR8 was recently shown to be selectively upregulated in intratumoral Treg cells and negatively correlates with survival of BC, CRC, and NSCLC patients (see for example Refs. 21, 22). The CCL1-CCR8 axis is also reported to potentiate the suppressive abilities of Treg cells (See for example Ref. 28). Extending these recent reports, results herein show that CCR8 is primarily expressed by peripheral blood Treg II rather than Treg I or III cells in patients with BC, and accordingly Treg II cells have stronger migratory potential towards CCL1. These results support CCL1-CC8 mediated recruitment of peripheral blood Treg II cells into human breast tumors.

Other chemokines are also involved in Treg cell recruitment into tumors. Increased frequencies of CCR2+ or CCR4+ intratumoral Treg cells have been reported in squamous cell carcinoma and melanoma, respectively (See for example Refs. 13, 29). In ovarian cancer, CCR4, CCR10, and CXCR3 have been reported to be involved in Treg recruitment into tumors. (See for example Refs. 30-32) Disruption of CCR5-dependent Treg cell infiltration was reported to inhibit pancreatic tumor growth (See for example Ref. 33). Our finding that CCR4, CCR5, and CXCR6 are also upregulated on peripheral blood Treg II and intratumoral Treg cells raise the importance of these additional chemokine receptors for recruitment into human breast tumors. In contrast, levels of CCR2, CCR10 and CXCR3 in peripheral blood Treg II cells were lower than in Tconv cells and these chemokine receptors were not selectively expressed on intratumoral T cells from patients with BC. Together with the findings that Treg II cells have similar expression patterns of several important immunoregulatory proteins, such as CD39, CTLA4, TIGIT, ICOS and OX40, with paired intratumoral Treg cells, our data support the notion that peripheral blood Treg II cells represent a major source of intratumoral Treg cells in patients with BC. This link is further strengthened by the finding that intratumoral Treg cells share more TCR clonal overlap with peripheral blood Treg II cells than with Treg I or III cells. In fact, intratumoral Treg cells share nearly no overlap with Treg I cells, suggesting that peripheral blood Treg I cells may not infiltrate into human breast tumors at all. Partial TCR clonal overlap between intratumoral Treg and Treg III cells suggests that Treg III cells may also infiltrate tumors, even though FoxP3 expression and immunosuppressive activity of Treg III cells in peripheral blood is low. It is possible that upon tumor infiltration, Treg III cells may further upregulate FoxP3 expression and other immunosuppressive molecules within the TME.

The composition of intratumoral Treg cells may also include induced Treg (iTreg) cells differentiated from FoxP3− naïve or effector CD4+ T cells locally within the TME, driven by tumor-derived factors (See for example Refs. 34, 35). While it has been shown that iTreg cells induced via TCR stimulation and TGFβ have very limited suppressive activity in vitro See for example Refs. 36, 37), it is possible that additional tumor-derived factors or direct interactions between iTreg cells with other cells within the TME may further enhance the immunosuppressive activity of iTreg cells in vivo. Lastly, it is possible that Treg II cells in peripheral blood may actually originate from tumors instead of the other way around as these are non-mutually exclusive possibilities.

Treg cells respond to a diverse range of cytokines. TGFβ-induced Smad2/3 activation promotes differentiation of peripherally-derived Treg (pTreg) cells and survival of thymus-derived Treg (tTreg) cells (See for example Refs. 38-40). TGFβ signaling is also required for the suppressive capability of Treg cells (See for example Ref. 41). IL-10 is known to stabilize Treg cell function through maintaining FoxP3 expression and amplifying IL-10 production (See for example Refs. 42,43). In contrast, Th2 cytokine IL-4 inhibits pTreg cell differentiation (See for example Refs. 44, 45) while Th1 cytokine IFNγ reduces the suppressive capacity of Treg cells and induces Treg cell fragility (See for example Ref. 46). Data herein shows that the suppressive potential of Treg cells may be indicated by the balance of their signaling responses to immunosuppressive vs. Th1/2 cytokines, which could be integrated via a cytokine signaling index (CSI). This notion that higher CSI indicates stronger suppressive potential was supported by the finding that higher Treg II CSI of patients with BC at diagnosis correlates with worse clinical outcome in two independent cohorts, lower intratumoral CD8/Treg ratio, and further supported by the positive correlation between CSI and in vitro suppression capacity of Treg II cells.

Within the TME, it has been shown that Treg cells can induce immunosuppressive M2 activation of TAMs and interactions between Treg cells and TAMs promote the development of an immunosuppressive TME (See for example Refs. 47, 48). Data herein shows that the percentage of Treg cells within 20 μm from TAMs, an indicator of their possible interaction within tumors, is higher in primary tumors from patients who later relapsed than patients who remained relapse-free, and that this is independent of Treg cell density within tumors. Furthermore, this relationship reflects cytokine signaling responsiveness in peripheral blood Treg II cells, but not Treg I or III cells. Data herein shows that Treg II cells increases CCL2 production by monocytes, which may act as a positive feedback loop to further increase monocytes/TAMs infiltration into tumors via producing more CCL2. These data suggest that peripheral blood Treg II cells with higher CSI may be more potent in interacting with TAMs within the TME. Furthermore, Treg-TAM interactions contribute to the development of an immunosuppressive TME via attracting more TAM infiltration in a feed forward cycle.

In summary, results herein demonstrate that peripheral blood Treg II cell subpopulation represents a major source of intratumoral Treg cells in human breast tumors, their cytokine signaling responsiveness reflects intratumoral immunosuppressive potential and predicts clinical outcome. The results also demonstrate that depleting peripheral blood Treg II cells might represent a promising immunotherapeutic approach for the treatment of BC.

Material and Methods

Human Samples

Peripheral blood mononuclear cells (PBMCs) were isolated from heparinized blood by Ficoll-Paque density centrifugation and cryopreserved in 10% DMSO FBS. Fresh breast tumors tissues were separated from fat tissues and minced into pieces up to 2 mm in diameter with scalpel blades. Single-cell suspensions were prepared by using the gentleMACS Dissociator (Miltenyl Biotec, Auburn, Calif., USA) according to the standard protocol. Tissue homogenates were treated with 0.26 Wunsch U/ml Liberase and 10 U/ml DNase (Sigma-Aldrich, St. Louis, Mo., USA) for up to 1 h as needed. The digested tissue homogenates were then filtered through a 100 μm filter.

Cytokine Stimulation Before Phosphoflow Cytometry

Cryopreserved PBMCs were thawed and rested for 16 hours. PBMCs were stimulated with IL-2 (100 U/ml), IL-10 (100 ng/ml), IFNγ (50 ng/ml), IL-4 (50 ng/ml) for 15 minutes or with TGFβ (25 ng/ml) for 30 mins (Peprotech, Rocky Hills, N.J., USA) at 37° C. followed by fixation with 1.5% paraformaldehyde (PFA) for 10 minutes at room temperature. Cells were washed with PBS to remove PFA, and permeabilized by the addition of 100% methanol. Cytokine-induced phosphorylations of STAT1/3/5/6 or Smad2/3 were then determined by phosphoflow cytometry. Further experimental details relating phosflow cytometry are described in the Life Sciences Reporting Summary.

Quantitative Immunofluorescence Image Analysis

The X and Y coordinates of the center of each cell's nucleus were acquired by inForm® Cell Analysis software (PerkinElmer). The percentages of FoxP3+ cells within 40 pixels (20 μm) to neighbor cells of a particular phenotype were determined by the K-nearest neighbor algorithm using R version 3.4.3.

TCR Sequencing

Intratumoral Treg cells (CD4+CD25+CD127−) were sorted from breast tumor tissue homogenates and autologous peripheral Treg cell subpopulation I, II or III cells were sorted from PBMCs isolated from 10 ml peripheral blood from BC patient. Genomic DNA was purified from sorted cells using DNA Micro Kit (Qiagen, Germantown, Md., USA). Amplification and sequencing of TCRβ CDR3 was performed using the immunoSEQ Platform (Adaptive Biotechnologies, Seattle, Wash., USA).

Statistical Analysis

Friedman tests were used to determine the statistical significance among peripheral Tconv and Treg cell subpopulations (Graphpad Prism, GraphPad Software, LaJolla, Calif., USA). Relapse-free survival (RFS) was defined as the time from the date of diagnosis of BC to the date of cancer recurrence or death. Kaplan-Meier method with log-rank test was used to determine cytokine signaling responsiveness as prognostic factors for RFS of patients with BC. All tests with p value <0.05 were considered statistically significant.

### REFERENCES

1. Josefowicz S Z, Lu L F, Rudensky A Y. Regulatory T cells: mechanisms of differentiation and function. Annual review of immunology 2012, 30: 531-564.
2. Lu L, Barbi J, Pan F. The regulation of immune tolerance by FOXP3. Nature reviews Immunology 2017, 17(11): 703-717.
3. Sakaguchi S, Miyara M, Costantino C M, Hafler D A. FOXP3+ regulatory T cells in the human immune system. Nature reviews Immunology 2010, 10(7): 490-500.
4. Kumar V. Adenosine as an endogenous immunoregulator in cancer pathogenesis: where to go? Purinergic signalling 2013, 9(2): 145-165.
5. Vignali D A, Collison L W, Workman C J. How regulatory T cells work. Nature reviews Immunology 2008, 8(7): 523-532.
6. Tanaka A, Sakaguchi S. Regulatory T cells in cancer immunotherapy. Cell research 2017, 27(1): 109-118.
7. Chaudhary B, Elkord E. Regulatory T Cells in the Tumor Microenvironment and Cancer Progression: Role and Therapeutic Targeting. Vaccines 2016, 4(3).
8. Bates G J, Fox S B, Han C, Leek R D, Garcia J F, Harris A L, et al. Quantification of regulatory T cells enables the identification of high-risk breast cancer patients and those at risk of late relapse. Journal of clinical oncology: official journal of the American Society of Clinical Oncology 2006, 24(34): 5373-5380.
9. Shang B, Liu Y, Jiang S J, Liu Y. Prognostic value of tumor-infiltrating FoxP3+ regulatory T cells in cancers: a systematic review and meta-analysis. Scientific reports 2015, 5: 15179.
10. Rech A J, Mick R, Martin S, Recio A, Aqui N A, Powell D J, Jr., et al. CD25 blockade depletes and selectively reprograms regulatory T cells in concert with immunotherapy in cancer patients. Science translational medicine 2012, 4(134): 134ra162.
11. Liakou C I, Kamat A, Tang D N, Chen H, Sun J, Troncoso P, et al. CTLA-4 blockade increases IFNgamma-producing CD4+ICOShi cells to shift the ratio of effector to regulatory T cells in cancer patients. Proceedings of the National Academy of Sciences of the United States of America 2008, 105(39): 14987-14992.
12. Ladoire S, Arnould L, Apetoh L, Coudert B, Martin F, Chauffert B, et al. Pathologic complete response to neoadjuvant chemotherapy of breast carcinoma is associated with the disappearance of tumor-infiltrating foxp3+ regulatory T cells. Clinical cancer research: an official journal of the American Association for Cancer Research 2008, 14(8): 2413-2420.
13. Sugiyama D, Nishikawa H, Maeda Y, Nishioka M, Tanemura A, Katayama I, et al. Anti-CCR4 mAb selectively depletes effector-type FoxP3+CD4+ regulatory T cells, evoking antitumor immune responses in humans. Proceedings of the National Academy of Sciences of the United States of America 2013, 110(44): 17945-17950.
14. Miyara M, Yoshioka Y, Kitoh A, Shima T, Wing K, Niwa A, et al. Functional delineation and differentiation dynamics of human CD4+ T cells expressing the FoxP3 transcription factor. Immunity 2009, 30(6): 899-911.
15. Liyanage U K, Moore T T, Joo H G, Tanaka Y, Herrmann V, Doherty G, et al. Prevalence of regulatory T cells is increased in peripheral blood and tumor microenvironment of patients with pancreas or breast adenocarcinoma. Journal of immunology 2002, 169(5): 2756-2761.
16. Martens A, Wistuba-Hamprecht K, Geukes Foppen M, Yuan J, Postow M A, Wong P, et al. Baseline Peripheral Blood Biomarkers Associated with Clinical Outcome of Advanced Melanoma Patients Treated with Ipilimumab. Clinical cancer research: an official journal of the American Association for Cancer Research 2016, 22(12): 2908-2918.
17. Yao X, Ahmadzadeh M, Lu Y C, Liewehr D J, Dudley M E, Liu F, et al. Levels of peripheral CD4(+)FoxP3(+) regulatory T cells are negatively associated with clinical response to adoptive immunotherapy of human cancer. Blood 2012, 119(24): 5688-5696.
18. Chinen T, Kannan A K, Levine A G, Fan X, Klein U, Zheng Y, et al. An essential role for the IL-2 receptor in Treg cell function. Nature immunology 2016, 17(11): 1322-1333.
19. Fontenot J D, Rasmussen J P, Gavin M A, Rudensky A Y. A function for interleukin 2 in Foxp3-expressing regulatory T cells. Nature immunology 2005, 6(11): 1142-1151.
20. Antonioli L, Pacher P, Vizi E S, Hasko G. CD39 and CD73 in immunity and inflammation. Trends in molecular medicine 2013, 19(6): 355-367.
21. Plitas G, Konopacki C, Wu K, Bos P D, Morrow M, Putintseva E V, et al. Regulatory T Cells Exhibit Distinct Features in Human Breast Cancer. Immunity 2016, 45(5): 1122-1134.
22. De Simone M, Arrigoni A, Rossetti G, Gruarin P, Ranzani V, Politano C, et al. Transcriptional Landscape of Human Tissue Lymphocytes Unveils Uniqueness of Tumor-Infiltrating T Regulatory Cells. Immunity 2016, 45(5): 1135-1147.
23. Campbell D J, Koch M A. Phenotypical and functional specialization of FOXP3+ regulatory T cells. Nature reviews Immunology 2011, 11(2): 119-130.
24. Vu M D, Xiao X, Gao W, Degauque N, Chen M, Kroemer A, et al. OX40 costimulation turns off Foxp3+ Tregs. Blood 2007, 110(7): 2501-2510.
25. Carstens J L, Correa de Sampaio P, Yang D, Barua S, Wang H, Rao A, et al. Spatial computation of intratumoral T cells correlates with survival of patients with pancreatic cancer. Nature communications 2017, 8: 15095.
26. Baras A S, Drake C, Liu J J, Gandhi N, Kates M, Hoque M O, et al. The ratio of CD8 to Treg tumor-infiltrating lymphocytes is associated with response to cisplatin-based neoadjuvant chemotherapy in patients with muscle invasive urothelial carcinoma of the bladder. Oncoimmunology 2016, 5(5): e1134412.
27. Hendry S, Salgado R, Gevaert T, Russell P A, John T, Thapa B, et al. Assessing Tumor-infiltrating Lymphocytes in Solid Tumors: A Practical Review for Pathologists and Proposal for a Standardized Method From the International Immunooncology Biomarkers Working Group: Part 1: Assessing the Host Immune Response, TILs in Invasive Breast Carcinoma and Ductal Carcinoma In Situ, Metastatic Tumor Deposits and Areas for Further Research. Advances in anatomic pathology 2017, 24(5): 235-251.
28. Barsheshet Y, Wildbaum G, Levy E, Vitenshtein A, Akinseye C, Griggs J, et al. CCR8(+)FOXp3(+) Treg cells as master drivers of immune regulation. Proc Natl Acad Sci USA 2017, 114(23): 6086-6091.
29. Loyher P L, Rochefort J, Baudesson de Chanville C, Hamon P, Lescaille G, Bertolus C, et al. CCR2 Influences T Regulatory Cell Migration to Tumors and Serves as a Biomarker of Cyclophosphamide Sensitivity. Cancer Res 2016, 76(22): 6483-6494.
30. Curiel T J, Coukos G, Zou L, Alvarez X, Cheng P, Mottram P, et al. Specific recruitment of regulatory T cells in ovarian carcinoma fosters immune privilege and predicts reduced survival. Nat Med 2004, 10(9): 942-949.
31. Facciabene A, Peng X, Hagemann I S, Balint K, Barchetti A, Wang L P, et al. Tumour hypoxia promotes tolerance and angiogenesis via CCL28 and T(reg) cells. Nature 2011, 475(7355): 226-230.
32. Redjimi N, Raffin C, Raimbaud I, Pignon P, Matsuzaki J, Odunsi K, et al. CXCR3+T regulatory cells selectively accumulate in human ovarian carcinomas to limit type I immunity. Cancer Res 2012, 72(17): 4351-4360.
33. Tan M C, Goedegebuure P S, Belt B A, Flaherty B, Sankpal N, Gillanders W E, et al. Disruption of CCR5-dependent homing of regulatory T cells inhibits tumor growth in a murine model of pancreatic cancer. J Immunol 2009, 182(3): 1746-1755.
34. Adeegbe D O, Nishikawa H. Natural and induced T regulatory cells in cancer. Frontiers in immunology 2013, 4: 190.
35. Su S, Liao J, Liu J, Huang D, He C, Chen F, et al. Blocking the recruitment of naive CD4(+) T cells reverses immunosuppression in breast cancer. Cell research 2017, 27(4): 461-482.
36. Walker M R, Carson B D, Nepom G T, Ziegler S F, Buckner J H. De novo generation of antigen-specific CD4+CD25+ regulatory T cells from human CD4+CD25− cells. Proceedings of the National Academy of Sciences of the U.S. Pat. No. 2,005,102(11): 4103-4108.
37. Tran D Q, Ramsey H, Shevach E M. Induction of FOXP3 expression in naive human CD4+FOXP3 T cells by T-cell receptor stimulation is transforming growth factor-beta dependent but does not confer a regulatory phenotype. Blood 2007, 110(8): 2983-2990.
38. Marie J C, Letterio J J, Gavin M, Rudensky A Y. TGF-beta1 maintains suppressor function and Foxp3 expression in CD4+CD25+ regulatory T cells. The Journal of experimental medicine 2005, 201(7): 1061-1067.
39. Schlenner S M, Weigmann B, Ruan Q, Chen Y, von Boehmer H. Smad3 binding to the foxp3 enhancer is dispensable for the development of regulatory T cells with the exception of the gut. The Journal of experimental medicine 2012, 209(9): 1529-1535.
40. Ouyang W, Beckett O, Ma Q, Li M O. Transforming growth factor-beta signaling curbs thymic negative selection promoting regulatory T cell development. Immunity 2010, 32(5): 642-653.
41. Konkel J E, Zhang D, Zanvit P, Chia C, Zangarle-Murray T, Jin W, et al. Transforming Growth Factor-beta Signaling in Regulatory T Cells Controls T Helper-17 Cells and Tissue-Specific Immune Responses. Immunity 2017, 46(4): 660-674.
42. Murai M, Turovskaya O, Kim G, Madan R, Karp C L, Cheroutre H, et al. Interleukin 10 acts on regulatory T cells to maintain expression of the transcription factor Foxp3 and suppressive function in mice with colitis. Nat Immunol 2009, 10(11): 1178-1184.
43. Chaudhry A, Samstein R M, Treuting P, Liang Y, Pils M C, Heinrich J M, et al. Interleukin-10 signaling in regulatory T cells is required for suppression of Th17 cell-mediated inflammation. Immunity 2011, 34(4): 566-578.
44. Wei J, Duramad O, Perng O A, Reiner S L, Liu Y J, Qin F X. Antagonistic nature of T helper 1/2 developmental programs in opposing peripheral induction of Foxp3+ regulatory T cells. Proc Natl Acad Sci USA 2007, 104(46): 18169-18174.
45. Caretto D, Katzman S D, Villarino A V, Gallo E, Abbas A K. Cutting edge: the Th1 response inhibits the generation of peripheral regulatory T cells. J Imunol 2010, 184(1): 30-34.
46. Overacre-Delgoffe A E, Chikina M, Dadey R E, Yano H, Brunazzi E A, Shayan G, et al. Interferon-gamma Drives Treg Fragility to Promote Anti-tumor Immunity. Cell 2017, 169(6): 1130-1141 e1111.
47. Sun W, Wei F Q, Li W J, Wei J W, Zhong H, Wen Y H, et al. A positive-feedback loop between tumour infiltrating activated Treg cells and type 2-skewed macrophages is essential for progression of laryngeal squamous cell carcinoma. British journal of cancer 2017, 117(11): 1631-1643.
48. Tiemessen M M, Jagger A L, Evans H G, van Herwijnen M J, John S, Taams L S. CD4+CD25+Foxp3+ regulatory T cells induce alternative activation of human monocytes/macrophages. Proceedings of the National Academy of Sciences of the United States of America 2007, 104(49): 19446-19451.

### P-EMBODIMENTS

### Embodiment P-1

A method of detecting cytokine signaling responsiveness in immune cells from a cancer subject, the method comprising:
(i) isolating cells from a blood sample from said cancer subject thereby forming an isolated blood cell fraction comprising isolated blood sample cells;
(ii) mixing the isolated blood sample cells with a cytokine, wherein said cytokine is selected from TGFβ, IL-10, IL-4 and IFNγ; and
(iii) detecting the responsiveness of the isolated blood sample cells to said cytokine.

### Embodiment P-2

The method of Embodiment P-1, wherein said detecting the responsiveness comprises quantifying an amount of responsiveness of the isolated blood sample cells to said cytokine.

### Embodiment P-3

The method of Embodiment P-2, comprising comparing the amount to a standard control, wherein if the amount is higher than said standard control, said cancer subject has a high risk of cancer relapse.

### Embodiment P-4

The method of Embodiment P-2, comprising comparing the amount to a standard control, wherein if the amount is lower than said standard control, said cancer subject has a high risk of cancer relapse.

### Embodiment P-5

The method of any one of Embodiment P-1 to P-4, wherein the cancer is selected from breast, melanoma, or gastrointestinal cancer.

### Embodiment P-6

The method of Embodiment P-5, wherein the cancer is breast cancer.

### Embodiment P-7

The method of any one of Embodiment P-1 to P-6, wherein the isolated blood cell fraction comprises leukocytes.

### Embodiment P-8

The method of Embodiment P-7, wherein the leukocytes are selected from lymphocytes and monocytes.

### Embodiment P-9

The method of Embodiment P-8, wherein the lymphocyte is a T-cell.

### Embodiment P-10

The methods of Embodiment P-9, wherein the T-cell is a Treg II cell.

### Embodiment P-11

The method of any one of Embodiment P-1 to P-10, wherein step (ii) further comprises mixing the isolated blood sample cells with one more more cytokines selected from IL-2, IL-3, IL-5, IL-6, IL-7, IL-8, IL-9, IL-11, IL-12, IL-13, IL-15, C-NTF, LIF, OSM (Oncostatin-M), EPO (Erythropoietin), G-CSF (GCSF), GH (Growth Hormone), PRL (Prolactin), IFN-alpha, IFN-beta, GM-CSF, M-CSF, SCF, IFN-gamma, IL1-alpha, IL1-beta, aFGF (FGF-acidic), bFGF (FGF-basic), INT-2, KGF (FGF7), EGF, TGF-alpha, Betacellulin (BTC), SCDGF, Amphiregulin, TNF-beta, PDGF, and HB-EGF.

### Embodiment P-12

A method of determining risk of relapse of cancer in a subject, comprising:
(i) isolating cells from a blood sample from said subject thereby forming an isolated blood cell fraction comprising isolated blood sample cells;
(ii) mixing the isolated blood sample cells with a cytokines, wherein said cytokine is selected from TGFβ, IL-10, IL-4 and IFNγ;
(iii) detecting the responsiveness of the isolated blood sample cells to said cytokine; and
(iv) determining whether said subject has a high risk of cancer relapse.

### Embodiment P-13

The method of Embodiment P-12, wherein the said detecting the responsiveness comprises quantifying an amount of responsiveness of the isolated blood sample cells to said cytokine.

### Embodiment P-14

The method of anyone of Embodiment P-12 to P-13, comprising comparing the amount to a standard control, wherein if the amount is higher than said standard control, said cancer subject has a high risk of cancer relapse.

### Embodiment P-15

The method of anyone of Embodiment P-12 to P-13, comprising comparing the amount to a standard control, wherein if the amount is lower than said standard control, said cancer subject has a high risk of cancer relapse.

### Embodiment P-16

The method of any one of Embodiment P-12 to P-15, wherein if said subject has a high risk of relapse, said patient is more closely monitored for cancer relapse and/or selected for more aggressive therapy.

### Embodiment P-17

The method of anyone of Embodiment P-12 to P-16, wherein the cancer is selected from breast, melanoma, or gastrointestinal cancer

### Embodiment P-18

The method of anyone of Embodiment P-12 to P-17, wherein the cancer is breast cancer.

### Embodiment P-19

The method of anyone of Embodiments P-12 to P-18, wherein the isolated blood cell fraction comprises leukocytes.

### Embodiment P-20

The method of Embodiment P-19, wherein the leukocytes are selected from lymphocytes and monocytes.

### Embodiment P-21

The method of Embodiment P-20, wherein the lymphocyte is a T-cell.

### Embodiment P-22

The methods of Embodiment P-21, wherein the T-cell is a Treg II cell.

### Embodiment P-23

The method of anyone of Embodiment P-12 to P-22, wherein step (ii) further comprises mixing the isolated blood sample cells with a cytokine selected from IL-2, IL-3, IL-5, IL-6, IL-7, IL-8, IL-9, IL-1, IL-12, IL-13, IL-15, C-NTF, LIF, OSM (Oncostatin-M), EPO (Erythropoietin), G-CSF (GCSF), GH (Growth Hormone), PRL (Prolactin), IFN-alpha, IFN-beta, GM-CSF, M-CSF, SCF, IFN-gamma, IL1-alpha, IL1-beta, aFGF (FGF-acidic), bFGF (FGF-basic), INT-2, KGF (FGF7), EGF, TGF-alpha, Betacellulin (BTC), SCDGF, Amphiregulin, TNF-beta, PDGF, and HB-EGF.

What is claimed is:

1. A method of detecting cytokine signaling responsiveness in immune cells from a cancer subject, the method comprising:
(i) isolating cells from a blood sample from said cancer subject thereby forming an isolated blood cell fraction comprising isolated blood sample cells;
(ii) mixing the isolated blood sample cells with a cytokine, wherein said cytokine is selected from TGFβ, IL-10, IL-4 and IFNγ; and
(iii) detecting the responsiveness of the isolated blood sample cells to said cytokine.

2. The method of claim 1, wherein said detecting the responsiveness comprises quantifying an amount of responsiveness of the isolated blood sample cells to said cytokine.

3. The method of claim 2, wherein the quantifying comprises calculating a cytokine signaling index.

4. The method of claim 3, wherein said cytokine signaling index is used to determine whether said cancer subject is at risk of cancer relapse.

5. The method of claim 1, wherein the cancer is selected from breast, melanoma, or gastrointestinal cancer.

6. The method of claim 5, wherein the cancer is breast cancer.

7. The method of claim 1, wherein the isolated blood cell fraction comprises leukocytes.

8. The method of claim 7, wherein the leukocytes are selected from lymphocytes and monocytes.

9. The method of claim 8, wherein the lymphocyte is a T-cell.

10. The methods of claim 9, wherein the T-cell is a Treg II cell.

11. The method of claim 1, wherein step (ii) further comprises mixing the isolated blood sample cells with one more cytokines selected from IL-2, IL-3, IL-5, IL-6, IL-7, IL-8, IL-9, IL-11, IL-12, IL-13, IL-15, C-NTF, LIF, OSM (Oncostatin-M), EPO (Erythropoietin), G-CSF (GCSF), GH (Growth Hormone), PRL (Prolactin), IFN-alpha, IFN-beta, GM-CSF, M-CSF, SCF, IFN-gamma, IL1-alpha, IL1-beta, aFGF (FGF-acidic), bFGF (FGF-basic), INT-2, KGF (FGF7), EGF, TGF-alpha, Betacellulin (BTC), SCDGF, Amphiregulin, TNF-beta, PDGF, and HB-EGF.

12. A method of determining risk of relapse of cancer in a subject, comprising:
(i) isolating cells from a blood sample from said subject thereby forming an isolated blood cell fraction comprising isolated blood sample cells;
(ii) mixing the isolated blood sample cells with a cytokines, wherein said cytokine is selected from TGFβ, IL-10, IL-4 and IFNγ;
(iii) detecting the responsiveness of the isolated blood sample cells to said cytokine; and
(iv) determining whether said subject has a high risk of cancer relapse.

13. The method of claim 12, wherein the said detecting the responsiveness comprises quantifying an amount of responsiveness of the isolated blood sample cells to said cytokine.

14. The method of claim 12, comprising comparing the amount to a standard control, wherein if the amount is higher than said standard control, said cancer subject has a high risk of cancer relapse.

15. The method of claim 12, comprising comparing the amount to a standard control, wherein if the amount is lower than said standard control, said cancer subject has a high risk of cancer relapse.

16. The method of claim 12, wherein if said subject has a high risk of relapse, said patient is more closely monitored for cancer relapse and/or selected for more aggressive therapy.

17. The method of claim 12, wherein the cancer is selected from breast, melanoma, or gastrointestinal cancer

18. The method of claim 12, wherein the cancer is breast cancer.

19. The method of claim 12, wherein the isolated blood cell fraction comprises leukocytes.

20. The method of claim 19, wherein the leukocytes are selected from lymphocytes and monocytes.

21. The method of claim 20, wherein the lymphocyte is a T-cell.

22. The methods of claim 21, wherein the T-cell is a Treg II cell.

23. The method of claim 12, wherein step (ii) further comprises mixing the isolated blood sample cells with a cytokine selected from IL-2, IL-3, IL-5, IL-6, IL-7, IL-8, IL-9, IL-11, IL-12, IL-13, IL-15, C-NTF, LIF, OSM (Oncostatin-M), EPO (Erythropoietin), G-CSF (GCSF), GH (Growth Hormone), PRL (Prolactin), IFN-alpha, IFN-beta, GM-CSF, M-CSF, SCF, IFN-gamma, IL1-alpha, IL1-beta, aFGF (FGF-acidic), bFGF (FGF-basic), INT-2, KGF (FGF7), EGF, TGF-alpha, Betacellulin (BTC), SCDGF, Amphiregulin, TNF-beta, PDGF, and HB-EGF.

24. A method of treating a cancer in a subject in need thereof comprising:
(i) isolating cells from a blood sample from said subject thereby forming an isolated blood cell fraction comprising isolated blood sample cells;
(ii) mixing the isolated blood sample cells with a cytokine, wherein said cytokine is selected from TGFβ, IL-10, IL-4 and IFNγ;
(iii) detecting the responsiveness of the isolated blood sample cells to said cytokine; and
(iv) treating said subject with a therapeutic regimen.

25. The method of claim 24, further comprising determining whether said subject has a high risk of cancer relapse, prior to step (iv).

26. The method of claim 24, wherein the said detecting the responsiveness comprises quantifying an amount of responsiveness of the isolated blood sample cells to said cytokine.

27. The method of any one of claims 25-26, wherein the determining step comprises comparing the amount of responsiveness to a standard control, wherein if the amount is higher than said standard control, said cancer subject has a high risk of cancer relapse.

28. The method of any one of claims 25-27, wherein step determining comprises comparing the amount to a standard control, wherein if the amount is lower than said standard control, said cancer subject has a low risk of cancer relapse.

29. The method of any one of claims 25-28, wherein if said subject has a high risk of relapse, said patient is more closely monitored for cancer relapse and/or selected for more aggressive therapy.

30. The method of any one of claims 24-29, wherein the cancer is selected from breast, melanoma, or gastrointestinal cancer

31. The method of any one of claims 24-30, wherein the cancer is breast cancer.

32. The method of any one of claims 24-31, wherein the isolated blood cell fraction comprises leukocytes.

33. The method of claim 32, wherein the leukocytes are selected from lymphocytes and monocytes.

34. The method of claim 33, wherein the lymphocyte is a T-cell.

35. The methods of claim 34, wherein the T-cell is a Treg II cell.

36. The method of any one of claims 24-35, wherein step (ii) further comprises mixing the isolated blood sample cells with a cytokine selected from IL-2, IL-3, IL-5, IL-6, IL-7, IL-8, IL-9, IL-11, IL-12, IL-13, IL-15, C-NTF, LIF, OSM (Oncostatin-M), EPO (Erythropoietin), G-CSF (GCSF), GH (Growth Hormone), PRL (Prolactin), IFN-alpha, IFN-beta, GM-CSF, M-CSF, SCF, IFN-gamma, IL1-alpha, IL1-beta, aFGF (FGF-acidic), bFGF (FGF-basic), INT-2, KGF (FGF7), EGF, TGF-alpha, Betacellulin (BTC), SCDGF, Amphiregulin, TNF-beta, PDGF, and HB-EGF.

37. The method of any one of claims 24-36, wherein the aggressive therapeutic regimen comprises one or more of a chemotherapy combination cyclophosphamide, methotrexate, fluorouracil, adriamycin, and taxane.

38. The method of claim 37, wherein the chemotherapy combination is cyclophosphamide, methotrexate, and fluorouracil.

39. The method of claim 37, wherein the chemotherapy combination is cyclophosphamide, Adriamycin, and fluorouracil.

40. The method of claim 37, wherein the chemotherapy combination is adriamycin and cyclophosphamide.

41. The method of claim 37, wherein the chemotherapy combination is adriamycin, cyclophosphamide, and taxane.

42. The method of claim 37, wherein the chemotherapy combination is fluorouracil, adriamycin, and cyclophosphamide.

43. The method of claim 37, wherein the chemotherapy combination is fluorouracil, adriamycin, cyclophosphamide, and taxane.

44. A method of preparing a sample comprising:
(i) isolating cells from a blood sample from a subject thereby forming an isolated blood cell fraction comprising isolated blood sample cells;
(ii) mixing the isolated blood sample cells with a cytokine, wherein said cytokine is selected from TGFβ, IL-10, IL-4 and IFNγ;
(iii) detecting the responsiveness of the isolated blood sample cells to said cytokine; and
(iv) quantifying an amount of responsiveness of the isolated blood sample cells to said cytokine.
